(19) United States
(12) Patent Application Publication
(10) Pub. No.: US 2023/0049455 A1
(43) Pub. Date: Feb. 16, 2023

(54) A CAS9-PDBD BASE EDITOR PLATFORM WITH IMPROVED TARGETING RANGE AND SPECIFICITY

(71) Applicant: UNIVERSITY OF MASSACHUSETTS, Boston, MA (US)

(72) Inventors: Scot A. Wolfe, Winchester, MA (US); Pengpeng Liu, Worcester, MA (US); Kevin Luk, Worcester, MA (US)

(21) Appl. No.: 17/796,184

(22) PCT Filed: Jan. 29, 2021
(86) PCT No.: PCT/US2021/015731

§ 371 Date: Jul. 28, 2022

Related U.S. Application Data
(60) Provisional application No. 62/968,484, filed on Jan. 31, 2020.

(51) Int. Cl. C12N 9/22; C12N 15/11; C12N 15/62; C12N 9/78; C12N 15/90; C07K 14/195

(52) U.S. Cl. CPC C12N 9/22; C07K 14/195; C12N 9/78; C12N 15/11; C12N 15/62; C12N 15/907; C12Y 305/04001; C12Y 305/04002; C07K 2319/81; C12N 2310/20; C12N 2800/80

(57) ABSTRACT

RNA-guided programmable cytosine and adenine base editors are a powerful class of genome editing tool for the introduction of localized base transitions without generating a double-stranded DNA break. Base editors (BE) have an optimal window of activity relative to the PAM recognized by the Cas9 enzyme and these constructs are strand selective. Here we demonstrate that fusion of a programmable DNA-binding domain (pDBD) or another Cas9 orthologue to spCas9-BE, we can produce an RNA-programmable Cas9-BE-pDBD chimera or Cas9-BE-Cas9 chimeras with dramatically improved activities and increased targeting range. Cas9-pDBD or Cas9-Cas9 fusion base editors display an expanded targeting repertoire and achieve highly specific genome editing, which can be tailored to achieve extremely precise genome editing at nearly any genomic locus.

### STATEMENT OF GOVERNMENT INTEREST

This invention was made with government support under GM115911 awarded by the National Institutes of Health. The government has certain rights in the invention.

### FIELD OF THE INVENTION

The present invention is related to the field of gene editing. The use of the presently disclosed accessory pDBD and/or orthogonal Cas9 systems enhances gene editing rates and the position of editing within a target sequence. The improved CRISPR platform provides an efficient conversion of the target base, and for limiting the rate of “bystander” conversion of bases that would be undesirable, which could create unwanted mutations. These disclosed fusion systems should also allow higher specificity for the base editing process, such as reduced off-target editing.

### BACKGROUND

Cas9 (clustered regularly interspaced short palindromic repeats; CRISPR-associated system) may be part of a bacterial immune response to foreign nucleic acid introduction. The development of Type II CRISPR/Cas9 systems as programmable nucleases for genome engineering has been beneficial in the biomedical sciences. For example, a Cas9 platform has enabled gene editing in a large variety of biological systems, where both gene knockouts and tailor-made alterations are possible within complex genomes. The CRISPR/Cas9 system has the potential for application to gene therapy approaches for disease treatment, whether for the creation of custom, genome-edited cell-based therapies or for direct correction or ablation of aberrant genomic loci within patients.

The safe application of Cas9 in gene therapy requires exceptionally high precision to ensure that undesired collateral damage to the treated genome may be minimized or, ideally, eliminated. Numerous studies have outlined features of Cas9 that can drive editing promiscuity, and a number of strategies (e.g. truncated single-guide RNAs (sgRNAs), nickases and FokI fusions) have been developed that improve the precision of this system. However all of these systems still suffer from a degree of imprecision (cleavage resulting in lesions at unintended target sites within the genome).

However, what may be needed in the art are further improvements in gene editing targeting range and specificity to facilitate reliable clinical applications that require simultaneous efficient and accurate editing of multigigabase genomes in billions to trillions of cells, depending on the scope of genetic repair that may be needed for therapeutic efficacy.

### SUMMARY

The present invention is related to the field of gene editing. The use of the presently disclosed accessory pDBD and/or orthogonal Cas9 systems enhances gene editing rates and the position of editing within a target sequence. The improved CRISPR platform provides an efficient conversion of the target base, and for limiting the rate of “bystander” conversion of bases that would be undesirable, which could create unwanted mutations. These disclosed fusion systems should also allow higher specificity for the base editing process, such as reduced off-target editing, in particular for target sites within a genome that have near cognate or identical sequences (e.g. genes with close paralogs).

In one embodiment, the present invention contemplates a method, comprising; a) providing; i) a nucleic acid sequence encoding at least one mutated base pair; and ii) a fusion protein comprising a Cas9/sgRNA complex, a programmable DNA binding domain and an adenine base editor (ABE) protein or a cytosine base editor (CBE) protein; b) contacting said fusion protein with said mutated base pair; and c) reverting said base pair to a wild type base pair. In one embodiment, the fusion protein further comprises an adenine or cytidine deaminase protein. In one embodiment, the programmable DNA binding domain is a zinc finger protein (ZFP), a transcription activator-like effector (TALE) domain or an orthogonal nuclease-dead Cas9 (dCas9)/sgRNA complex. In one embodiment, the Cas9/sgRNA complex is a mutant (D10A) that nicks one DNA strand. In one embodiment, the at least one mutated base pair is an MECP2 gene mutation. In one embodiment, the method further provides a biological sample comprising said at least one mutated base pair. In one embodiment, the biological sample is a human biological sample. In one embodiment, the method further comprises administering said fusion protein to a patient exhibiting at least one symptom of a genetic disease. In one embodiment, the method further comprises reducing said at least one symptom of said genetic disease with said fusion protein. In one embodiment, the genetic disease is Rett syndrome. In one embodiment, the adenine base editor or said cytosine base editor hybridizes and forms an R-loop proximate to a protospacer adjacent motif (PAM) containing a single G. In one embodiment, the adenine base editor or said cytosine base editor hybridizes to a protospacer adjacent motif that is non-canonical for said Cas9/sgRNA complex. In one embodiment, the fusion protein is selected from the group consisting of a CBE/ABE-nSpyCas9-ZFP fusion protein, a CBE/ABE-nSpyCas9-TALE fusion protein and a CBE/ABE-nSpyCas9-dSauCas9/dNme2Cas9 fusion protein. In one embodiment, the fusion protein comprises a base conversion activity that has a two-fold greater efficiency than a standard CBE/ABE Cas9 system.

In one embodiment, the present invention contemplates a composition comprising a Cas9/sgRNA framework, a programmable DNA binding domain and an adenine base editor (ABE) protein or a cytosine base editor (CBE) protein that hybridizes proximate to a protospacer adjacent motif containing a single G. In one embodiment, the Cas9/sgRNA complex further comprises an adenine deaminase protein or a cytidine deaminase protein. In one embodiment, the programmable DNA binding domain is a zinc finger protein(ZFP), a transcription activator-like effector (TALE) domain or an orthogonal dCas9/sgRNA complex. In one embodiment, the Cas9 nickase component of the CBE/ABE has attenuated DNA binding affinity to a dual G containing protospacer adjacent motif. In one embodiment, the fusion protein comprises a base conversion activity that has a greater than two-fold reduction in off-target activity than a standard CBE/ABE Cas9 system.

In one embodiment, the present invention contemplates an attenuated Cas9 protein having a PAM recognition domain comprising at least two amino acid substitutions, wherein said PAM recognition domain has an attenuated affinity for its cognate PAM sequence.. In one embodiment, the at least two amino acid substitutions comprise R1333S and K1118S. In one embodiment, the at least two amino acid substitutions comprise R1335K and E1219Q. In one embodiment, the at least two amino acid substitutions comprise R1333S, E1219Q and K1118S. In one embodiment, the attenuated Cas9 protein is attached to a pDBD protein. In one embodiment, the pDBD protein is a zinc finger protein, a TALE or a dCas9 protein.

In one embodiment, the present invention contemplates a method, comprising; a) providing; i) a mutated nucleic acid sequence comprising a disease mutation; and ii) a Cas9/sgRNA complex attached to a programmable DNA binding domain, wherein said programmable DNA binding domain is an adenine or cytosine base editor protein; b) contacting said Cas9/sgRNA complex to said nucleic acid sequence; and c) reverting said mutated nucleic acid sequence to a wild type sequence. In one embodiment, the programmable DNA binding domain is selected from a zinc finger protein and an orthogonal dCas9/sgRNA complex. In one embodiment, the disease mutation is an MECP2 gene mutation. In one embodiment, the method further provides a biological sample comprising said mutated nucleic acid. In one embodiment, the biological sample is a human biological sample. In one embodiment, the method further comprises administering said Cas9/sgRNA complex to a patient exhibiting at least one symptom of a genetic disease. In one embodiment, the method further comprises reducing said at least one symptom of said genetic disease. In one embodiment, the genetic disease is Rett syndrome.

In one embodiment, the present invention contemplates a composition comprising a Cas9/sgRNA complex attached to a programmable DNA binding domain, wherein said programmable DNA binding domain is an adenine or a cytosine base editor protein. In one embodiment, the programmable DNA binding domain is selected from a zinc finger protein, TALE and an orthogonal dCas9/sgRNA complex.

### DEFINITIONS

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but also plural entities and also includes the general class of which a specific example may be used for illustration. The terminology herein may be used to describe specific embodiments of the invention, but their usage does not delimit the invention, except as outlined in the claims.

The term “about” as used herein, in the context of any of any assay measurements refers to +/- 5% of a given measurement.

As used herein, the term “CRISPRs” or “Clustered Regularly Interspaced Short Palindromic Repeats” refers to an acronym for DNA loci that contain multiple, short, direct repetitions of base sequences. Each repetition contains a series of bases followed by the same series in reverse and then by 30 or so base pairs known as “spacer DNA”. The spacers are short segments of DNA from a virus and may serve as a ‘memory’ of past exposures to facilitate an adaptive defense against future invasions (PMID 25430774).

As used herein, the term “Cas” or “CRISPR-associated (cas)” refers to genes often associated with CRISPR repeat-spacer arrays (PMID 25430774).

As used herein, the term “Cas9” refers to a nuclease from Type II CRISPR systems, an enzyme specialized for generating double-strand breaks in DNA, with two active cutting sites (the HNH and RuvC domains), one for each strand of the double helix. Jinek combined tracrRNA and spacer RNA into a “single-guide RNA” (sgRNA) molecule that, mixed with Cas9, could find and cleave DNA targets through Watson-Crick pairing between the guide sequence within the sgRNA and the target DNA sequence (PMID 22745249). There have been substantial efforts to broaden the targeting specificity of SpyCas9 through mutations that increase the number of PAMs that can be recognized. Two of the most prominent modified versions of Cas9 are xCas9 (Hu et al. 2018 (PMID 29512652)) and Cas9-NG (Nishimasu et al. 2018 (PMID 30166441)), both of which permit targeting some additional PAM elements.

As used herein, the term “nuclease deficient Cas9”, “nuclease dead Cas9” or “dCas9” refers to a modified Cas9 nuclease wherein the nuclease activity has been disabled by mutating residues in the RuvC and HNH catalytic domains. Disabling of both cleavage domains can convert Cas9 from a RNA-programmable nuclease into an RNA-programmable DNA recognition complex to deliver effector domains to specific target sequences (Qi, et al. 2013 (PMID 23452860) and Gilbert, et al. 2013 PMID 23849981) or to deliver an independent nuclease domain such as FokI. A nuclease dead Cas9 can bind to DNA via its PAM recognition domain and guide RNA, but will not cleave the DNA.

The term “nuclease dead Cas9 Fokl fusion” or “FokI-dCas9” as used herein, refers to a nuclease dead Cas9 that may be fused to the cleavage domain of Fokl, such that DNA recognition may be mediated by dCas9 and the incorporated guide RNA, but that DNA cleavage may be mediated by the Fokl domain (Tsai, et al. 2014 (PMID 24770325) and Guilinger, et al. (PMID 24770324)). Fokl normally requires dimerization in order to cleave the DNA, and as a consequence two FokI-dCas9 complexes must bind in proximity in order to cleave the DNA. Fokl can be engineer such that it functions as an obligate heterodimer.

As used herein, the term “catalytically active Cas9” refers to an unmodified Cas9 nuclease comprising full nuclease activity.

The term “nickase” as used herein, refers to a nuclease that cleaves only a single DNA strand, either due to its natural function or because it has been engineered to cleave only a single DNA strand. Cas9 nickase variants (e.g. nSpCas9, nCas9) that have either the RuvC or the HNH domain mutated provide control over which DNA strand is cleaved and which remains intact (Jinek, et al. 2012 (PMID 22745249) and Cong, et al. 2013 (PMID 23287718)).

The term “cytidine deaminase” refers to a protein domain that converts cytosine to uracil in the target DNA strand. In the context of a cytosine base editor, the cytidine deaminase drives the conversion of a C-G base pair to an T-A base pair. There are a large number of different cytidine deaminases that have been used in cytosine base editors - natural deaminases, such as rAPOBEC1, and engineered variants such as BE4 (Huang, et. al. 2021 (PMID 33462442) and references therein). The type of cytidine deaminase domain can be swapped within cytosine base editors to change the base conversion efficiency in different sequence contexts.

The term “adenine deaminase” refers to a protein domain that converts adenine to inosine in the target DNA strand. In the context of a adenine base editor, the adenine deaminase drives the conversion of an A-T base pair to a G-C base pair. There are a number of different adenine deaminases that have been evolved for use in adenine base editors, such as TadA7.10 and TadA8e (Huang, et. al. 2021 (PMID 33462442) and references therein). The type of adenine deaminase domain can be swapped within adenine base editors to change the base conversion efficiency in different sequence contexts.

The term “DNA targeting unit”, “DTU” as used herein, refers to any type of system that can be programmed to recognize a specific DNA sequence of interest. Such DNA targeting units can include, but are not limited to a “programmable DNA binding domain” (either called a pDBD or simply a DBD), as defined below, and/or a CRISPR/Cas9 or CRISPR/Cas12a (Cpf1) system that may be programmed by a RNA guide (either a single guide RNA or a crRNA and tracrRNA combination) to recognize a particular target site.

The term, “trans-activating crRNA”, “tracrRNA” as used herein, refers to a small trans-encoded RNA. For example, CRISPR/Cas (clustered, regularly interspaced short palindromic repeats/CRISPR-associated proteins) constitutes an RNA-mediated defense system, which protects against viruses and plasmids. This defensive pathway has three steps. First a copy of the invading nucleic acid is integrated into the CRISPR locus. Next, CRISPR RNAs (crRNAs) are transcribed from this CRISPR locus. The crRNAs are then incorporated into effector complexes, where the crRNA guides the complex to the invading nucleic acid and the Cas proteins degrade this nucleic acid. There are several pathways of CRISPR activation, one of which requires a tracrRNA, which plays a role in the maturation of crRNA. TracrRNA is complementary to base pairs with a pre-crRNA forming an RNA duplex. This is cleaved by RNase III, an RNA-specific ribonuclease, to form a crRNA/tracrRNA hybrid. This hybrid acts as a guide for the endonuclease Cas9, which cleaves the invading nucleic acid.

The term “programmable DNA binding domain” as used herein, refers to any protein comprising a pre-determined sequence of amino acids that bind to a specific nucleotide sequence. Such binding domains can include, but are not limited to, a zinc finger protein, a homeodomain and/or a transcription activator-like effector protein.

The term “protospacer adjacent motif” (or PAM) as used herein, refers to a DNA sequence that may be required for a Cas9/sgRNA to form an R-loop to interrogate a specific DNA sequence through Watson-Crick pairing of its guide RNA with the genome. The PAM may comprise a trinucleotide sequence having a single G residue (e.g., a single G PAM), or a trinucleotide sequence having two consecutive G residues (e.g., a dual G PAM). The PAM specificity may be a function of the DNA-binding specificity of the Cas9 protein (e.g., a “protospacer adjacent motif recognition domain” at the C-terminus of Cas9).

As used herein, the term “sgRNA” refers to single guide RNA used in conjunction with CRISPR associated systems (Cas). sgRNAs are a fusion of crRNA and tracrRNA and contain nucleotides of sequence complementary to the desired target site (Jinek, et al. 2012 (PMID 22745249)). Watson-Crick pairing of the sgRNA with the target site permits R-loop formation, which in conjunction with a functional PAM permits DNA cleavage or in the case of nuclease-deficient Cas9 allows binds to the DNA at that locus.

As used herein, the term “orthogonal” refers targets that are non-overlapping, uncorrelated, or independent. For example, if two orthogonal Cas9 isoforms were utilized, they would employ orthogonal sgRNAs that only program one of the Cas9 isoforms for DNA recognition and cleavage (Esvelt, et al. 2013 (PMID 24076762); Edraki, et. al 2018 (PMID 30581144)). For example, this would allow one Cas9 isoform (e.g. S. pyogenes Cas9 or SpCas9) to function as a nuclease or nickase programmed by a sgRNA that may be specific to it, and another Cas9 isoform (e.g. N. meningitidis Cas9, Nm1Cas9 or Nme2Cas9) to operate as a nuclease dead Cas9 that provides DNA targeting to a binding site through its PAM specificity and orthogonal sgRNA. Other Cas9s include S. aureus Cas9 or SaCas9 and A. naeslundii Cas9 or AnCas9.

As used herein, the term “methyl CpG binding protein 2 (MECEP2)” refers to a gene that encodes the protein MECP2. MECP2 appears to be essential for the normal function of nerve cells. The protein seems to be particularly important for mature nerve cells, where it is present in high levels. The MECP2 gene is located on the X chromosome.

As used herein, the term “Rett syndrome” refers to a disease caused by a mutation in an MECP2 gene.

The term “truncated” as used herein, when used in reference to either a polynucleotide sequence or an amino acid sequence means that at least a portion of the wild type sequence may be absent. In some cases truncated guide sequences within the sgRNA or crRNA may improve the editing precision of Cas9 (Fu, et al. 2014 (PMID 24463574)).

The term “dimerization domain” as used herein, refers to a domain, either protein, polynucleotide that allows the associate of two different molecules. A dimerization domain can allow homotypic and/or heterotypic interactions. Dimerization domains can also be drug-dependent (i.e. depending on the presence of a small molecule in order to function) (Liang, et al. (PMID 21406691) and Ho, et al. 1996 (PMID 8752278)).

The term “base pairs” as used herein, refer to specific nucleobases (also termed nitrogenous bases), that are the building blocks of nucleotide sequences that form a primary structure of both DNA and RNA. Double stranded DNA may be characterized by specific hydrogen bonding patterns, base pairs may include, but are not limited to, guanine-cytosine and adenine-thymine) base pairs.

The term “specific genomic target” as used herein, refers to any pre-determined nucleotide sequence capable of binding to a Cas9 protein contemplated herein. The target may include, but may be not limited to, a nucleotide sequence complementary to a programmable DNA binding domain or an orthogonal Cas9 protein programmed with its own guide RNA, a nucleotide sequence complementary to a single guide RNA, a protospacer adjacent motif recognition domain, an on-target binding sequence and an off-target binding sequence.

The term “on-target binding sequence” as used herein, refers to a subsequence of a specific genomic target that may be completely complementary to a programmable DNA binding domain and/or a single guide RNA sequence.

The term “off-target binding sequence” as used herein, refers to a subsequence of a specific genomic target that may be partially complementary to a programmable DNA binding domain and/or a single guide RNA sequence.

The term “bystander editing” or “bystander effect” as used herein refers to the conversion by an ABE or CBE of a nearby base pair that is not the target position where editing is desired (Huang, et. al. 2021 (PMID 33462442)). Such a bystander edit can result in an undesired mutation to a gene or a regulatory element that may alter the function of the gene or regulatory element in an undesired manner.

The term “fails to bind” as used herein, refers to any nucleotide-nucleotide interaction or a nucleotide-amino acid interaction that exhibits partial complementarity, but has insufficient complementarity for recognition to trigger the cleavage of the target site by the Cas9 nuclease. Such binding failure may result in weak or partial binding of two molecules such that an expected biological function (e.g., nuclease activity) fails.

The term “cleavage” as used herein, may be defined as the generation of a break in the DNA. This could be either a single-stranded break or a double-stranded break depending on the type of nuclease that may be employed.

As used herein, the term “edit” “editing” or “edited” refers to a method of altering a nucleic acid sequence of a polynucleotide (e.g., for example, a wild type naturally occurring nucleic acid sequence or a mutated naturally occurring sequence) by selective deletion of a specific genomic target, the specific inclusion of new sequence through the use of an exogenously supplied DNA template, or the conversion of one DNA base to another DNA base. Such a specific genomic target includes, but may be not limited to, a chromosomal region, mitochondrial DNA, a gene, a promoter, an open reading frame or any nucleic acid sequence.

The term “delete”, “deleted”, “deleting” or “deletion” as used herein, may be defined as a change in either nucleotide or amino acid sequence in which one or more nucleotides or amino acid residues, respectively, are, or become, absent.

As used herein, the terms “complementary” or “complementarity” are used in reference to “polynucleotides” and “oligonucleotides” (which are interchangeable terms that refer to a sequence of nucleotides) related by the base-pairing rules. For example, the sequence “C-A-G-T,” may be complementary to the sequence “A-C-T-G.” Complementarity can be “partial” or “total.” “Partial” complementarity may be where one or more nucleic acid bases may be not matched according to the base pairing rules. “Total” or “complete” complementarity between nucleic acids may be where each and every nucleic acid base may be matched with another base under the base pairing rules. The degree of complementarity between nucleic acid strands has significant effects on the efficiency and strength of hybridization between nucleic acid strands. This may be of particular importance in amplification reactions, as well as detection methods which depend upon binding between nucleic acids.

The terms “homology” and “homologous” as used herein in reference to nucleotide sequences refer to a degree of complementarity with other nucleotide sequences. There may be partial homology or complete homology (i.e., identity). A nucleotide sequence which may be partially complementary, i.e., “substantially homologous,” to a nucleic acid sequence may be one that at least partially inhibits a completely complementary sequence from hybridizing to a target nucleic acid sequence. The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous sequence to a target sequence under conditions of low stringency. This may be not to say that conditions of low stringency are such that non-specific binding may be permitted; low stringency conditions require that the binding of two sequences to one another be a specific (i.e., selective) interaction. The absence of non-specific binding may be tested by the use of a second target sequence which lacks even a partial degree of complementarity (e.g., less than about 30% identity); in the absence of non-specific binding the probe will not hybridize to the second non-complementary target.

The terms “homology” and “homologous” as used herein in reference to amino acid sequences refer to the degree of identity of the primary structure between two amino acid sequences. Such a degree of identity may be detected in a portion of each amino acid sequence, or to the entire length of the amino acid sequence. Two or more amino acid sequences that are “substantially homologous” may have at least 50% identity, preferably at least 75% identity, more preferably at least 85% identity, most preferably at least 95%, or 100% identity.

An oligonucleotide sequence which may be a “homolog” may be defined herein as an oligonucleotide sequence which exhibits greater than or equal to 50% identity to a sequence, when sequences having a length of 100 bp or larger are compared.

As used herein, the term “gene” means the deoxyribonucleotide sequences comprising the coding region of a structural gene and including sequences located adjacent to the coding region on both the 5’ and 3' ends for a distance of about 1 kb on either end such that the gene corresponds to the length of the full-length mRNA. The sequences which are located 5’ of the coding region and which are present on the mRNA are referred to as 5’ non-translated sequences. The sequences which are located 3’ or downstream of the coding region and which are present on the mRNA are referred to as 3’ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into heterogeneous nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

The term “gene of interest” as used herein, refers to any pre-determined gene for which deletion may be desired.

The term “allele” as used herein, refers to any one of a number of alternative forms of the same gene or same genetic locus.

The term “protein” as used herein, refers to any of numerous naturally occurring extremely complex substances (as an enzyme or antibody) that consist of amino acid residues joined by peptide bonds, contain the elements carbon, hydrogen, nitrogen, oxygen, usually sulfur. In general, a protein comprises amino acids having an order of magnitude within the hundreds.

The term “peptide” as used herein, refers to any of various amides that are derived from two or more amino acids by combination of the amino group of one acid with the carboxyl group of another and are usually obtained by partial hydrolysis of proteins. In general, a peptide comprises amino acids having an order of magnitude with the tens.

The term “polypeptide”, refers to any of various amides that are derived from two or more amino acids by combination of the amino group of one acid with the carboxyl group of another and are usually obtained by partial hydrolysis of proteins. In general, a peptide comprises amino acids having an order of magnitude with the tens or larger.

“Nucleic acid sequence” and “nucleotide sequence” as used herein refer to an oligonucleotide or polynucleotide, and fragments or portions thereof, and to DNA or RNA of genomic or synthetic origin which may be single- or double-stranded, and represent the sense or antisense strand.

The term “an isolated nucleic acid”, as used herein, refers to any nucleic acid molecule that has been removed from its natural state (e.g., removed from a cell and may be, in a preferred embodiment, free of other genomic nucleic acid).

The terms “amino acid sequence” and “polypeptide sequence” as used herein, are interchangeable and to refer to a sequence of amino acids.

As used herein the term “portion” when in reference to a protein (as in “a portion of a given protein”) refers to fragments of that protein. The fragments may range in size from four amino acid residues to the entire amino acid sequence minus one amino acid.

The term “portion” when used in reference to a nucleotide sequence refers to fragments of that nucleotide sequence. The fragments may range in size from 5 nucleotide residues to the entire nucleotide sequence minus one nucleic acid residue.

As used herein, the term “hybridization” may be used in reference to the pairing of complementary nucleic acids using any process by which a strand of nucleic acid joins with a complementary strand through base pairing to form a hybridization complex. Hybridization and the strength of hybridization (i.e., the strength of the association between the nucleic acids) may be impacted by such factors as the degree of complementarity between the nucleic acids, stringency of the conditions involved, the Tₘ of the formed hybrid, and the G:C ratio within the nucleic acids.

As used herein the term “hybridization complex” refers to a complex formed between two nucleic acid sequences by virtue of the formation of hydrogen bounds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution (e.g., C₀ t or R₀ t analysis) or between one nucleic acid sequence present in solution and another nucleic acid sequence immobilized to a solid support (e.g., a nylon membrane or a nitrocellulose filter as employed in Southern and Northern blotting, dot blotting or a glass slide as employed in in situ hybridization, including FISH (fluorescent in situ hybridization)).

As used herein, the term “Tₘ ” may be used in reference to the “melting temperature.” The melting temperature may be the temperature at which a population of double-stranded nucleic acid molecules becomes half dissociated into single strands. As indicated by standard references, a simple estimate of the Tₘ value may be calculated by the equation: Tₘ = 81.5 + 0.41 (% G+C), when a nucleic acid may be in aqueous solution at 1M NaCl. Anderson et al., “Quantitative Filter Hybridization” In: Nucleic Acid Hybridization (1985). More sophisticated computations take structural, as well as sequence characteristics, into account for the calculation of Tₘ.

As used herein the term “stringency” may be used in reference to the conditions of temperature, ionic strength, and the presence of other compounds such as organic solvents, under which nucleic acid hybridizations are conducted. “Stringency” typically occurs in a range from about Tₘ to about 20° C. to 25° C. below Tₘ. A “stringent hybridization” can be used to identify or detect identical polynucleotide sequences or to identify or detect similar or related polynucleotide sequences. For example, when fragments are employed in hybridization reactions under stringent conditions the hybridization of fragments which contain unique sequences (i.e., regions which are either non-homologous to or which contain less than about 50% homology or complementarity) are favored. Alternatively, when conditions of “weak” or “low” stringency are used hybridization may occur with nucleic acids that are derived from organisms that are genetically diverse (i.e., for example, the frequency of complementary sequences may be usually low between such organisms).

As used herein, the term “amplifiable nucleic acid” may be used in reference to nucleic acids which may be amplified by any amplification method. It may be contemplated that “amplifiable nucleic acid” will usually comprise “sample template.”

As used herein, the term “sample template” refers to nucleic acid originating from a sample which may be analyzed for the presence of a target sequence of interest. In contrast, “background template” may be used in reference to nucleic acid other than sample template which may or may not be present in a sample. Background template may be most often inadvertent. It may be the result of carryover, or it may be due to the presence of nucleic acid contaminants sought to be purified away from the sample. For example, nucleic acids from organisms other than those to be detected may be present as background in a test sample.

“Amplification” may be defined as the production of additional copies of a nucleic acid sequence and may be generally carried out using polymerase chain reaction. Dieffenbach C. W. and G. S. Dveksler (1995) In: PCR Primer, a Laboratory Manual, Cold Spring Harbor Press, Plainview, N.Y.

As used herein, the terms “restriction endonucleases” and “restriction enzymes” refer to bacterial enzymes, each of which cut double-stranded DNA at or near a specific nucleotide sequence.

DNA molecules are said to have “5’ ends” and “3’ ends” because mononucleotides are reacted to make oligonucleotides in a manner such that the 5’ phosphate of one mononucleotide pentose ring may be attached to the 3’ oxygen of its neighbor in one direction via a phosphodiester linkage. Therefore, an end of an oligonucleotide may be referred to as the “ 5’ end” if its 5’ phosphate may be not linked to the 3' oxygen of a mononucleotide pentose ring. An end of an oligonucleotide may be referred to as the “3’ end” if its 3’ oxygen may be not linked to a 5’ phosphate of another mononucleotide pentose ring. As used herein, a nucleic acid sequence, even if internal to a larger oligonucleotide, also may be said to have 5' and 3' ends. In either a linear or circular DNA molecule, discrete elements are referred to as being “upstream” or 5’ of the “downstream” or 3’ elements. This terminology reflects the fact that transcription proceeds in a 5' to 3' fashion along the DNA strand. The promoter and enhancer elements which direct transcription of a linked gene are generally located 5’ or upstream of the coding region. However, enhancer elements can exert their effect even when located 3’ of the promoter element and the coding region. Transcription termination and polyadenylation signals are located 3’ or downstream of the coding region.

As used herein, the term “an oligonucleotide having a nucleotide sequence encoding a gene” means a nucleic acid sequence comprising the coding region of a gene, i.e. the nucleic acid sequence which encodes a gene product. The coding region may be present in a cDNA, genomic DNA or RNA form. When present in a DNA form, the oligonucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the terms “nucleic acid molecule encoding”, “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

The term “bind”, “binding”, or “bound” as used herein, includes any physical attachment or close association, which may be permanent or temporary. Generally, an interaction of hydrogen bonding, hydrophobic forces, van der Waals forces, covalent and ionic bonding etc., facilitates physical attachment between the molecule of interest and the analyte being measuring. The “binding” interaction may be brief as in the situation where binding causes a chemical reaction to occur. That may be typical when the binding component may be an enzyme and the analyte may be a substrate for the enzyme. Reactions resulting from contact between the binding agent and the analyte are also within the definition of binding for the purposes of the present invention.

### BRIEF DESCRIPTION OF THE FIGURES

The file of this patent contains at least one drawing executed in color. Copies of this patent with color drawings will be provided by the Patent and Trademark Office upon request and payment of the necessary fee.

FIG. 1 illustrates several types of base editors (BEs) described in this disclosure. (top) Adenine or cytosine base editors (ABE or CBE) composed of an SpyCas9 nickase (nSpyCas9) fused to a programmable DNA-binding domain (pDBD). The star indicates mutations in the PAM recognition residues that render SpyCas9 recognition dependent on the attached pDBD. The red circle indicates the strand that is directly modified by base editing. (bottom) Adenine or cytosine base editors (ABE or CBE) composed of an SpyCas9 nickase fused to a nuclease dead orthogonal Cas9 (dCas9), such as Nme2Cas9.

FIG. 2 illustrates several embodiments of CBE variants based on a BE4 framework¹⁸ with modifications to the nuclear localization signal (NLS) sequences to improve their nuclear localization. Slotted into the dotted position within the top construct are the various CBE platforms that were tested.

FIG. 3 presents exemplary data showing Relative positions of the ZFP (Zif268), TALE, dSauCas9 and dNme2Cas9 binding sites in the KANK3, PLXNB2 and TGM2 loci. All of the TALE binding sites (green) are on the top strand. The Zif268 binding sites (red) in the TGM2 and PLXNB2 binding sites are on the complementary strand to the indicated regions. PAM sequences for the SauCas9 (NNGRRT) and Nme2Cas9 (NNNNCC) are indicated (brown/magenta).

FIG. 4 presents exemplary data of an aggregate heat map of CBE editing rates across 10 target sites containing an NGG PAM for the SpCas9 recognition element. The activity range scale is shown to the left of the heat map. A low level of indels (not C to T conversion events) was detected for all of the samples, the average of which is indicated in numbers on the far right side of the panel. The D1 and D2 nomenclatures indicate the two possible relative orientations of the Cas9-Cas9 binding sites, where D1 is recognition of opposite strands and D2 is recognition of the same strand. In all cases the SpCas9 recognition domain avoids overlap with the attached pDBD or orthogonal dCas9 binding site. The numbering scheme at the top indicates the position of the C relative to the PAM, where C1 is most distal from the PAM. Data are from biological triplicate experiments characterized by Illumina sequencing.

FIG. 5 presents exemplary data shown an aggregate heat map of CBE editing rates across 17 target sites containing an NGH PAM for the SpCas9 recognition element (H = A, C or T). The activity range scale is shown to the left of the heat map. A low level of indels (not C to T conversion events) was detected for all of the samples, the average of which is indicated in numbers on the far right side of the panel. The D1 and D2 nomenclatures indicate the two possible relative orientations of the Cas9-Cas9 binding sites, where D1 is recognition of opposite strands and D2 is recognition of the same strand. In all cases the SpCas9 recognition domain avoids overlap with the attached pDBD or orthogonal dCas9 binding site. The numbering scheme at the top indicates the position of the C relative to the PAM, where C1 is most distal from the PAM. Data are from biological triplicate experiments characterized by Illumina sequencing.

FIG. 6 presents exemplary data showing an aggregate heat map of CBE editing rates across 15 target sites containing an NHG PAM for the SpCas9 recognition element. The activity range scale is shown to the left of the heat map. A low level of indels (not C to T conversion events) was detected for all of the samples, the average of which is indicated in numbers on the far right side of the panel. The D1 and D2 nomenclatures indicate the two possible relative orientations of the Cas9-Cas9 binding sites, where D1 is recognition of opposite strands and D2 is recognition of the same strand. In all cases the SpCas9 recognition domain avoids overlap with the attached pDBD or orthogonal dCas9 binding site. The numbering scheme at the top indicates the position of the C relative to the PAM, where C1 is most distal from the PAM. Data are from biological triplicate experiments characterized by Illumina sequencing

FIGS. 7A and 7B present exemplary data showing activity profiles of SpyCas9 BE4 (gray bars) relative to SpCas9- dSauCas9 BE4 (A) or SpCas9-dNme2Cas9 BE4 (B) across the KANK3 locus. C to T conversion activity is indicated for 18 different target sites (TS#), where the bp number indicates the rough separation distance between the Cas9-Cas9 binding sites. Activities are shown for both the D1 and D2 orientation of Cas9-Cas9 binding sites (color indicated in panel legend). Note that the active target sites for SpCas9 BE4 are those with the NGG PAMs for SpCas9 (denoted by black dots below TS#). The black arrow indicates the presence of enhancement in base editing rates even 139 bp distant from the dSauCas9 binding site.

FIG. 8 presents exemplary data showing an activity profile of SpyCas9 BE4 (SpCas9, gray bars) relative to SpCas9-zif268 BE4 (SpCas9-Zif, red) across the KANK3 locus. C to T conversion activity is indicated for 18 different target sites (TS#), where the bp number indicates the rough separation between the Cas9-ZFP binding sites. Note that the active target sites for SpCas9 BE4 are those with the NGG PAMs for SpCas9 (denoted by black dots below TS#). The other sites contain NGH or NHG PAMs. The black arrow indicates the presence of enhancement in nSpyCas9 base editing rates 97 bp distant from the ZFP binding site.

FIG. 9 presents an illustrative schematic diagram of a SpyCas9 ABE. R-loop formation between the guide and the target sequence liberates one genomic DNA stand for base editing. A short segment of the single-strand DNA (Base conversion Window) is appropriately positioned and accessible to a fused adenine deaminase module. A to G conversion in the sequence (and T to C on the opposite strand) is driven via DNA repair by a nick introduced by Cas9 on the opposite DNA strand.

FIG. 10 presents an exemplary target site overview of five common MECP2 mutations. Local sequence surrounding common C>T pathogenic mutations in MECP2, where the position of the mutation (bold T) and the resulting amino acid change is noted. Coding strand is top strand. Bold A indicates the target base for deamination. The nearest base conversion window targetable by a standard SpyCas9 ABE (SpABE) based on the presence of an NGG PAM is indicated with a red bar. Only two of the 5 target adenines fall within this window. The position of the non-standard PAM utilized by our proposed SpCas9-DBD ABE fusion (*ABE) is underlined and indicated in brackets below the sequence [5’->3’]. All of these targets are accessible, and in all cases position the target A at the center of the window where base conversion rates are expected to be maximal.

FIG. 11 provides exemplary data showing an efficient base conversion of C to T at non-standard PAMs by SpyCas9-DBD cytidine deaminase. A SpyCas9 cytosine BE fused to a DBD was programmed with different guides (magenta boxes) to target neighboring regions of a gene to “walk” across the locus utilizing different non-standard PAMs (blue boxes). The SpyCas9-DBD BE was delivered by transient transfection into cells and after 3 days the population of cells was harvested and their genomic DNA amplified and sequenced to assess the rate of C to T conversion. All of the non-standard PAMs achieved functional C to T conversion (peaks indicated by *). Only the standard nGG PAM was functional with standard SpyCas9 BE (data not shown).

FIG. 12 presents an illustrative schematic overview of Cas9-DBD frameworks. SpyCas9 is fused to a DNA-binding domain (DBD) — either a Zinc finger protein (ZFP) or a nuclease-dead orthogonal Cas9 (dCas9) - that recognizes a neighboring sequence to the SpyCas9 target site. The DBD subunit delivers the Cas9 to the target region of the genome, which allows it to function at non-standard PAMs that have low affinity. Once R-loop formation is initiated the PAM element does not impact SpyCas9 catalytic activity. These SpyCas9-DBD systems increase the number of targetable sequences for Cas9, and can also increase the specificity of their activity within the genome.

FIG. 13 presents an illustrative schematic of a CRISPR adenine base editor reporter.

FIG. 14 presents exemplary data showing the quantification of a CRISPR adenine base editing rates at different PAM sequences.

FIG. 15 presents an illustrative schematic of a CRISPR cytosine base editor reporter

FIG. 16 presents exemplary data showing the quantification of a CRISPR cytosine base editing rates at different PAM sequences.

FIG. 17 presents illustrative schematics showing enhanced CBEs and ABEs. The dotted rectangles in the main constructs indicate the position where each Cas9 or Cas9-fusion variant was positioned in the construct. Examples of the constructs are displayed below. These examples are not exhaustive.

FIG. 18 presents exemplary data showing the improved activity of an enhanced Cas9/cytosine base editor.

FIG. 18A: An illustrative design of a CBE CopGFP reporter used to evaluate the activity of CBE constructs. Conversion of the H mutation to Y, red, restores green fluorescence; Spacer sequence, underlined; PAM, blue. Target sequence, red.

FIG. 18B: Quantification of CBE efficacy by calculating % GFP⁺ cells. Base editors used were nSpCas9, nSpCas9-NG, nxCas9, nSpCas9-dSaCas9, and nSpCas9-dNme2Cas9 fused with BE4. The “n” prefix before the name of the SpCas9 version indicates the D10A nickase. Negative = no DNA control.

FIG. 19 presents exemplary data showing the improved activity of an enhanced Cas9/cytosine base editor having a TGT PAM sequence. Base editing was performed with nSpCas9, nSpCas9-NG, nxCas9, nSpCas9-Zif268, nSpCas9-TALE, nSpCas9-dSaCas9, and nSpCas9-dNme2Cas9 fused with BE4 in HEK293T cells. Data is dispalyed as a heatmap of C-to-T editing frequencies induced by enhanced CBE systems at KANK3 TS1 (PAM = TGT). Intensity of square reflects the mean of three independent biological replicates.

FIG. 20 presents exemplary data showing the improved activity of an enhanced Cas9/cytosine base editor having a AGT PAM sequence. Base editing was performed with nSpCas9, nSpCas9-NG, nxCas9, nSpCas9-Zif268, nSpCas9-TALE, nSpCas9-dSaCas9, and nSpCas9-dNme2Cas9 fused with BE4 in HEK293T cells. Data is displayed as a heatmap of C-to-T editing frequencies induced by enhanced CBE systems at KANK3 TS2 (PAM = ATG). Intensity of square reflects the mean of three independent biological replicates.

FIG. 21 presents exemplary data showing the improved activity of an enhanced Cas9/cytosine base editor having a TGA PAM sequence. Base editing was performed with nSpCas9, nSpCas9-NG, nxCas9, nSpCas9-Zif268, nSpCas9-TALE, nSpCas9-dSaCas9, and nSpCas9-dNme2Cas9 fused with BE4 in HEK293T cells. Data is displayed as a heatmap of C-to-T editing frequencies induced by enhanced CBE systems at KANK3 TS3 (PAM = TGA). Intensity of square reflects the mean of three independent biological replicates.

FIG. 22 presents exemplary data showing the improved activity of an enhanced Cas9/cytosine base editor having a GTG PAM sequence. Base editing was performed with nSpCas9, nSpCas9-NG, nxCas9, nSpCas9-Zif268, nSpCas9-TALE, nSpCas9-dSaCas9, and nSpCas9-dNme2Cas9 fused with BE4 in HEK293T cells. Data is displayed as a heatmap of C-to-T editing frequencies induced by enhanced CBE systems at KANK3 TS4 (PAM = GTG). Intensity of square reflects the mean of three independent biological replicates.

FIG. 23 presents exemplary data showing the improved activity of an enhanced Cas9/cytosine base editor having a GGG PAM sequence. Base editing was performed with nSpCas9, nSpCas9-NG, nxCas9, nSpCas9-Zif268, nSpCas9-TALE, nSpCas9-dSaCas9, and nSpCas9-dNme2Cas9 fused with BE4 in HEK293T cells. Data is displayed as a heatmap of C-to-T editing frequencies induced by enhanced CBE systems at KANK3 TS5 (PAM = GGG). Intensity of square reflects the mean of three independent biological replicates.

FIG. 24 presents exemplary data showing the improved activity of an enhanced Cas9/adenine base editor.

FIG. 24A: An illustrative design of an ABE mCherry reporter used to evaluate the activity of each ABE construct. STOP codon, red, conversion to G1n codon restores mCherry signal; Spacer sequence, underlined; PAM, blue, target site, red, is on the complementary strand.

FIG. 24B: Quantification of ABE efficacy by calculating % mCherry+ cells. Base editors used were nSpCas9, nSpCas9-NG, nxCas9, nSpCas9-dSaCas9, and nSpCas9-dNme2Cas9 fused to ABEmax. The “n” prefix before the name indicates the D10A nickase. Negative, no DNA control.

FIG. 25 presents exemplary data showing the improved activity of an enhanced Cas9/adenine base editor having a TGT PAM sequence. Base editing was performed with nSpCas9, nSpCas9-NG, nxCas9, nSpCas9-Zif268, nSpCas9-TALE, nSpCas9-dSaCas9, and nSpCas9-dNme2Cas9 fused with ABE7.10 in HEK293T cells. Data is displayed as a heatmap of A-to-G editing frequencies induced by enhanced ABE systems at KANK3 TS1 (PAM = TGT). Intensity of square reflects the mean of three independent biological replicates.

FIG. 26 presents exemplary data showing the improved activity of an enhanced Cas9/adenine base editor having an ATG PAM sequence. Base editing was performed with nSpCas9, nSpCas9-NG, nxCas9, nSpCas9-Zif268, nSpCas9-TALE, nSpCas9-dSaCas9, and nSpCas9-dNme2Cas9 fused with ABE7.10 in HEK293T cells. Data is displayed as a heatmap of A-to-G editing frequencies induced by enhanced ABE systems at KANK3 TS2 (PAM = ATG). Intensity of square reflects the mean of three independent biological replicates.

FIG. 27 presents exemplary data showing the improved activity of an enhanced Cas9/adenine base editor having an TGA PAM sequence. Base editing was performed with nSpCas9, nSpCas9-NG, nxCas9, nSpCas9-Zif268, nSpCas9-TALE, nSpCas9-dSaCas9, and nSpCas9-dNme2Cas9 fused with ABE7.10 in HEK293T cells. Data is displayed as a heatmap of A-to-G editing frequencies induced by enhanced ABE systems at KANK3 TS3 (PAM = TGA). Intensity of square reflects the mean of three independent biological replicates.

FIG. 28 presents exemplary data showing the improved activity of an enhanced Cas9/adenine base editor having an GTG PAM sequence. Base editing was performed with nSpCas9, nSpCas9-NG, nxCas9, nSpCas9-Zif268, nSpCas9-TALE, nSpCas9-dSaCas9, and nSpCas9-dNme2Cas9 fused with ABE7.10 in HEK293T cells. Data is displayed as a heatmap of A-to-G editing frequencies induced by enhanced ABE systems at KANK3 TS4 (PAM = GTG). Intensity of square reflects the mean of three independent biological replicates.

FIG. 29 presents exemplary data showing the improved activity of an enhanced Cas9/adenine base editor having an GGG PAM sequence. Base editing was performed with nSpCas9, nSpCas9-NG, nxCas9, nSpCas9-Zif268, nSpCas9-TALE, nSpCas9-dSaCas9, and nSpCas9-dNme2Cas9 fused with ABE7.10 in HEK293T cells. Data is displayed as a heatmap of A-to-G editing frequencies induced by enhanced ABE systems at KANK3 TS5 (PAM = GGG). Intensity of square reflects the mean of three independent biological replicates.

FIG. 30 presents exemplary data showing the improved activity of an enhanced Cas9/adenine base editor having an NGG PAM sequence. Data is displayed as a heatmap depicting the summary of the base editing frequency at each adenine in the spacer region for ten guide RNAs targeting sites with NGG PAMs using nSpCas9, nSpCas9-NG, nxCas9, nSpCas9-Zif268, nSpCas9-TALE, nSpCas9-dSaCas9, and nSpCas9-dNme2Cas9 fused with ABE7.10 in HEK293T cells. Values and intensity of square reflect the mean of three independent biological replicates.

FIG. 31 presents exemplary data showing the improved activity of an enhanced Cas9/adenine base editor having an NGH PAM sequence. Data is displayed as a heatmap depicting the summary of the base editing frequency at each adenine in the spacer region for 14 guide RNAs targeting sites with NGH PAMs using nSpCas9, nSpCas9-NG, nxCas9, nSpCas9-Zif268, nSpCas9-TALE, nSpCas9-dSaCas9, and nSpCas9-dNme2Cas9 fused with ABE7.10 in HEK293T cells. Values and intensity of square reflect the mean of three independent biological replicates.

FIG. 32 presents exemplary data showing the improved activity of an enhanced Cas9/adenine base editor having an NHG PAM sequence. Data is displayed as a heatmap depicting the summary of the base editing frequency at each adenine in the spacer region for 14 guide RNAs targeting sites with NHG PAMs using nSpCas9, nSpCas9-NG, nxCas9, nSpCas9-Zif268, nSpCas9-TALE, nSpCas9-dSaCas9, and nSpCas9-dNme2Cas9 fused with ABE7.10 in HEK293T cells. Values and intensity of square reflect the mean of three independent biological replicates.

FIG. 33 presents exemplary constructs of attenuated Cas9 proteins. The schematic shows enhanced CBEs and ABEs comprising an SpCas9 with multiple amino acid substitutions to further attenuate cognate cleavage activity in the absence of a fused DNA targeting unit such as a ZFP, as opposed to a wild type SpCas9 protein or a single amino acid substituted SpCas9 protein (e.g., R1333S or R1335S). The dotted rectangles in the main constructs indicate the position where each Cas9 or Cas9-fusion variant was positioned in the construct. Examples of the constructs are displayed below. These examples are not exhaustive.

FIG. 34 presents exemplary data showing the improvement in non-cognate base editing subsequent to attachment of a pDBD (e.g., ZFP) to a Cas9 protein. The data compares adenine base editing frequency between wild type nSpCas9, attenuated nSpCas9^{R1333S,K1118S} and attenuated nSpCas9^{R1335K,E1219Q} fused to the TadA8e domain with (+) or without (-) Zif268 targeting KANK3 TS1-TS5. The PAM for each target site is indicated above each set of bars.

FIG. 35 presents data demonstrating the dependence of the attenuated Cas9 base editor on the attached pDBD for target site editing. The activity of the nSpCas9^{R1335K,E1219Q,K1118S} fused to the TadA8e domain was tested with and without the pDBD (zinc finger protein Zif268) at the KANK3 locus. In the absence of the pDBD, sanger sequencing of the genomic DNA of the population of treated cells indicates that there was minimal conversion of the adenines on the complementary strand (positions highlighted by red boxes), which would be read out as T to C conversion on the sequenced strand. In the presence of the Zif268 fusion, the SpCas9 ABE causes base conversion at two of the three highlighted positions. Numeral grad below indicates the estimate of each base at each DNA position based on the chromatogram. Control = untreated genomic DNA.

### DETAILED DESCRIPTION OF THE INVENTION

The present invention is related to the field of gene editing. The use of the presently disclosed accessory pDBD and/or orthogonal Cas9 systems enhances gene editing rates and the position of editing within a target sequence. The improved CRISPR platform provides an efficient conversion of the target base, and for limiting the rate of “bystander” conversion of bases that would be undesirable, which could create unwanted mutations. These disclosed fusion systems should also allow higher specificity for the base editing process, such as reduced off-target editing.

### I. Cytosine And Adenine Base Editor Proteins

Genome editing systems have been developed from these systems were recently described: cytosine^{1,2} and adenine³ base editors. These systems allow the conversion of cytosine to thymine or adenine to guanine within the DNA. These base editor systems can be used to revert point mutations⁴, introduce stop codons⁵, disrupt splicing sequences⁶, all of which can be used for therapeutic applications. One challenge with the current Cas9 base editing systems is the necessity to have a complementary PAM at the correct position and on the appropriate DNA strand to target the activity of the cytosine or adenosine base editors to precise genomic positions that are targeted for conversion, as base editors usually are strand-specific with regards to their activity. Consequently, there have been substantial efforts to broaden the targeting specificity of SpyCas9 through mutations that increase the number of PAMs that can be recognized. Two of the most prominent modified versions of Cas9 are xCas97 and Cas9-NG8 , both of which permit targeting some additional PAM elements.

A new class of genome editing systems developed from CRISPR/Cas9 systems were recently described: cytosine (Komor, et. al. 2016 (PMID 27096365) and Nishida, et al. 2016 (PMID 27492474)) and adenine (Gaudelli, et al. 2017 (PMID 29160308) base editors (CBE/ABE). These base editors typically contain two components: the adenine or cytidine deaminase and the Cas9/sgRNA complex (or Cas12a/crRNA complex), where the Cas9 component is mutated so that it cannot produce a double-strand break. Typically the Cas9 component will be a strand specific nickase (e.g. D10A mutant of SpyCas9). These systems allow the strand-specific conversion of cytosine to uracil or adenine to guanine within the DNA (Huang, et. al. 2021 (PMID 33462442)). These base editor systems can be used to revert point mutations, introduce stop codons, disrupt splicing sequences, all of which can be valuable for therapeutic applications.

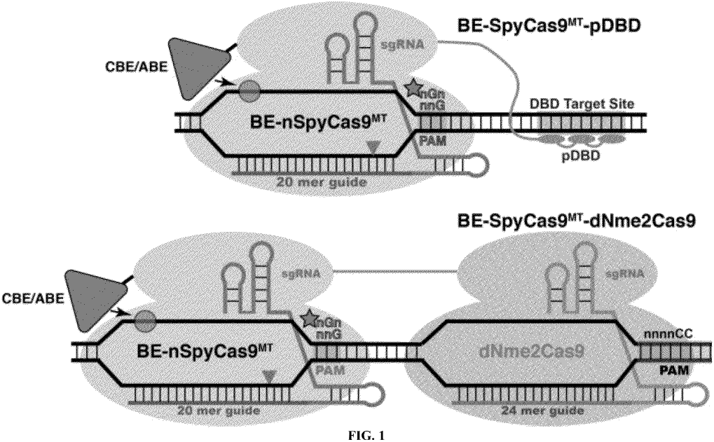

In one embodiment, the present invention contemplates a Cas9-base editing platform that has a much broader targeting range for PAM recognition than the standard SpyCas9 systems. For example, the Cas9-base editing platform hybridizes proximate to a single G (NGN or NNG) rather than two Gs as in traditional NGG SpyCas9 PAM motifs. FIG. 1. This was achieved by appending programmable DNA-binding domains (pDBD)⁹, such as zinc finger proteins (ZFP)¹⁰ or TALE domains¹¹, or an orthogonal dCas9¹². Orthogonal Cas9 variants (e.g., Nme2Cas9) recognize C- rich PAM motifs and work with these same fusion strategies¹³. This platform allows nucleic acid sequence targeting almost anywhere in in the genome on either strand, dramatically expanding the number of disease-causing mutations that can potentially be corrected via base editors.

Since these base editing systems are not dependent on a specific stage of the cell cycle for function, and require no accessory elements beside the programmed guide RNA, they are able to function efficiently in post-mitotic cells¹⁴. Other favorable aspects of Cas9-pDBD or Cas9-Cas9 fusion systems includes, but is not limited to, the attenuation of the PAM recognition binding affinity of SpCas9 to render DNA recognition dependent on the associated pDBD or nuclease dead orthogonal Cas9. It has been reported that the SpyCas9 nuclease can dramatically improve the specificity of the Cas9 nuclease^{9,12}. Since base editors can produce off-target DNA editing at near cognate target sequences within a genome^{7,15-17}, the ability to limit the activity of the base editor to the DNA target sequence can provide many advantages that are compatible with the presently disclosed adenine-cytosine based editing Cas9 fusion systems.

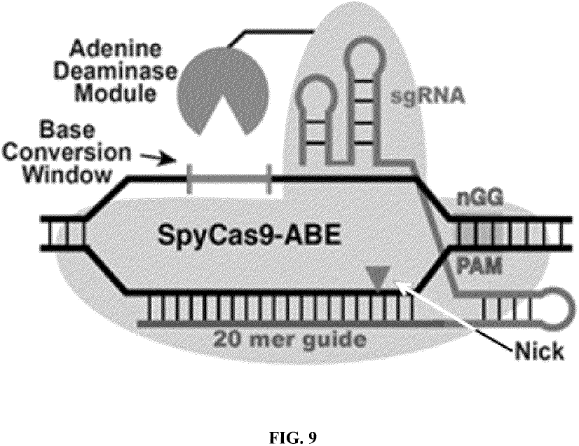

SpyCas9 base editors have been developed that facilitate the site-specific transition of cytosine to thymine (C to T) or adenine to guanine (A to G, which achieves T to C) within a specific genomic locus. 18, 39-40 The SpyCas9 base editing systems are believed to achieve base conversion by delivering a cytosine or adenine deaminase module to a specific genomic region where they can act on the single-stranded DNA region that is created upon Cas9 R-loop formation with its target sequence. FIG. 9. Fixation of the mutation within the genome is facilitated through the generation of a nick in the non- edited DNA strand.³⁹ These base editor systems are functional in vivo in post-mitotic cells,⁴¹ and do not require the production of a double strand break (DSB) to institute sequence modification, which mitigates the production of some forms of collateral DNA damage associated with nuclease-based DSB generation.⁴² These conventional CBE and ABE gene editors have a primary disadvantage of not being validated as a base editor for each specific mutation of interest.

### II. CRISPR CBE and ABE Gene Editing Platforms

CRISPR-Cas9-based genome editing systems have revolutionized genome editing approaches and are now being leveraged for a broad range of commercial and therapeutic applications. The present invention contemplates embodiments comprising a CRISPR platform integrated with CBE and/or ABE gene editing platforms comprising an enhanced activity and targeting range as compared to other previously reported CRISPR systems.

In one embodiment, the present invention contemplates compositions comprising a cytosine base editing (CBE) and/or an adenine base editing (ABE) platform including, but not limited to, CBE/ABE-nSpyCas9-ZFP fusions, CBE/ABE-nSpyCas9-TALE and CBE/ABE-nSpyCas9-dSauCas9/dNme2Cas9 frameworks. Although it is not necessary to understand the mechanism of an invention, it is believed that such CBE and ABE platforms can be used for efficient and specific base conversion in a variety of sequence contexts. The data included herein demonstrate the successful creation of robust CBE platforms in the CBE-nSpyCas9-ZFP fusions, CBE-nSpyCas9-TALE and CBE-nSpyCas9- dSauCas9/dNme2Cas9 frameworks that can target a far broader range of DNA sequences with higher efficiency than existing frameworks (e.g., SpyCas9, xCas9 or Cas9-NG). In one embodiment, the present invention contemplates a method for targeting disease alleles in patient-derived cell lines to examine the potential clinical efficacy of these systems with the presently disclosed CBE and ABE base editing CRISPR platforms. Although it is not necessary to understand the mechanism of an invention, it is believed that the presently disclosed CBE and ABE base editing CRISPR platforms may provide a therapeutic application for efficient base conversion in target tissue containing a pathogenic point mutation.

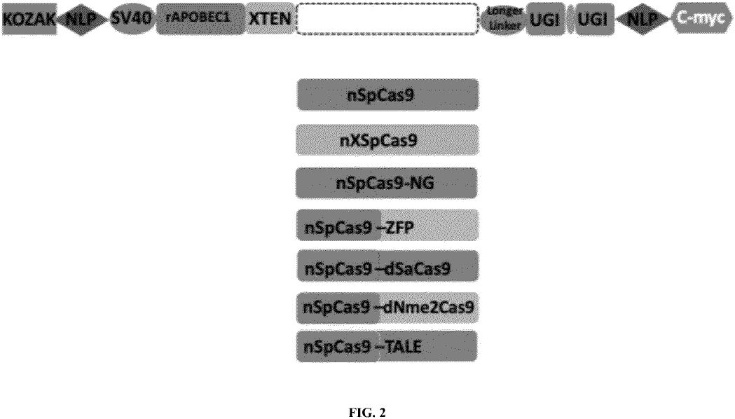

In one embodiment, the present invention contemplates a composition comprising a Cas9/sgRNA framework comprising a pDBD protein or a second Cas9 fusion protein integrated as an adenine base editor or a cytosine base editor (e.g., a BE4-based cytosine base editor¹⁸), wherein said base editor hybridizes proximate to a single G protospacer adjacent motif. See, FIG. 2. In one embodiment, a wild- type SpyCas9 nickase (nSpyCas9) is used to create a fusion protein. The activity of these constructs were tested across nucleic acid loci (e.g., KANK3, PLNXB2 & TGM2) spanning 42 different target sites, where these target sites contained a variety of NGG, NGH and NHG PAMs (H = A, C or T) for the SpyCas9 recognition module.

This BE4-based cytosine base editor framework was compared to the nickase versions of the “wild-type” SpyCas9, xCas9⁷ and Cas9-NG⁸. These latter two systems have been shown to facilitate the recognition of a broader set of PAMs beyond the standard NGG PAM for SpyCas9. In one embodiment, the present invention contemplates a CBE-Cas9 framework comprising a zinc finger within the Cas9-ZFP fusion system. In one embodiment, the zinc finger is employing Zif268, which contains three zinc fingers and has a well defined 10 bp recognition motif²⁰ that is present in all three of the target loci¹⁹. For the Cas9-TALE constructs, an artificial TALE domain was generated for each tested nucleic acid loci using “golden gate” assembly methods²¹.

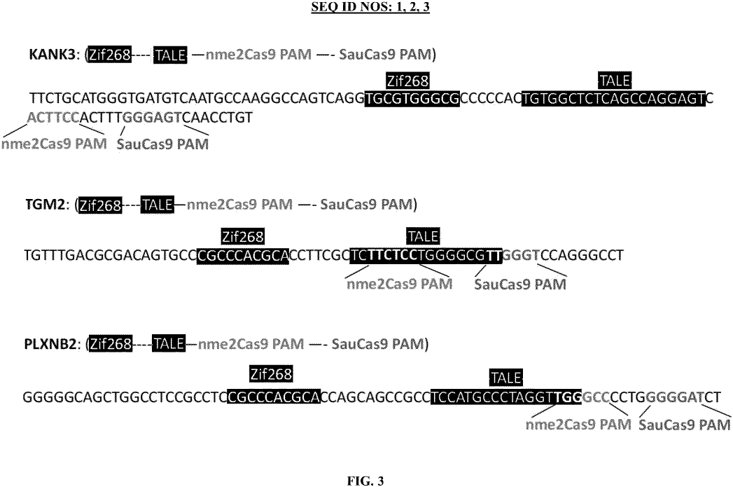

FIG. 3. In one embodiment, the present invention contemplates a composition comprising a Cas9-Cas9 fusion construct comprising an orthogonal nuclease-dead Cas9 (dCas9) with an sgRNA that is specific for each locus to anchor binding of the SpyCas9 nickase within the target locus. In one embodiment, the dCas9 comprises SauCas9²² or Nme2Cas9¹³. The presently disclosed data was performed in HEK293T cells by transient transfection of expression plasmids, with Illumina deep sequencing of PCR amplicons spanning the target site used for quantification of the editing rates.

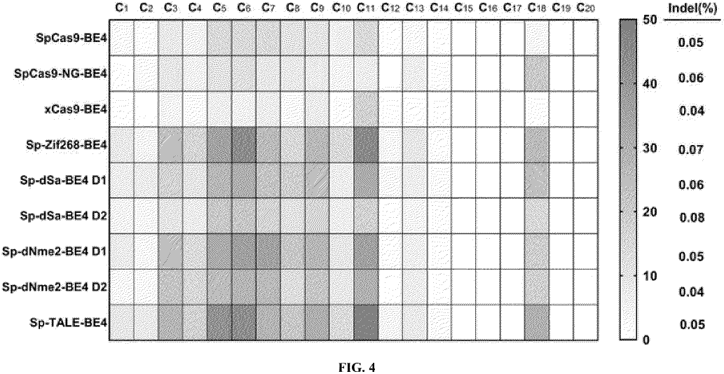

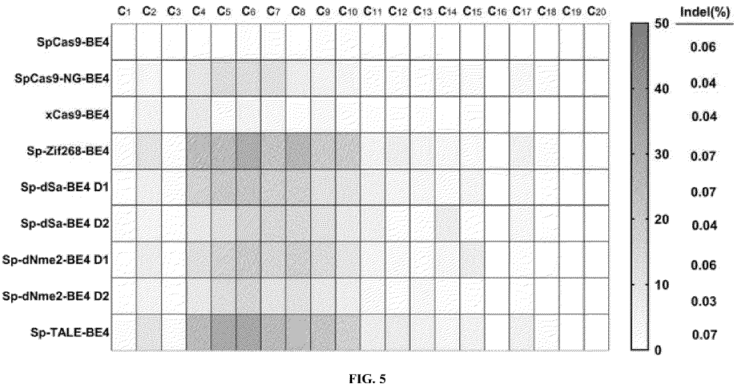

At canonical NGG PAM target sites all of the CBEs are functional, although the Cas9-pDBD and Cas9-Cas9 fusion proteins outperform the single Cas9 constructs (SpCas9, SpCas9-NG and xCas9) in most instances even at canonical target sites. FIG. 4. The Cas9- ZFP fusions and the Cas9-TALE fusions perform particularly well with regards to achieving higher base conversion activity in this assay. At non-canonical NGH PAM target sites the “wild-type” SpCas9 BE4 construct displays little activity. FIG. 5. The SpCas9-NG and xCas9 display modest activity, with the Cas9-NG construct proving to be the most robust of these two. The Cas9-Cas9 fusion proteins outperform the single Cas9 constructs in most instances - in particular for the D1 orientation of the target sites. The Cas9-ZFP fusions and the Cas9- TALE fusions perform particularly well with regards to higher base conversion activity at the NGH PAM target sites.

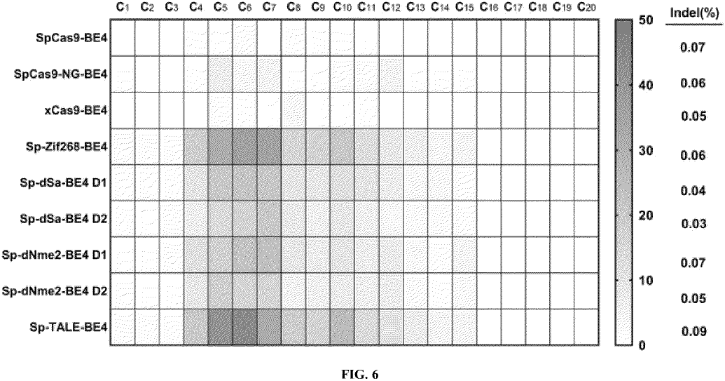

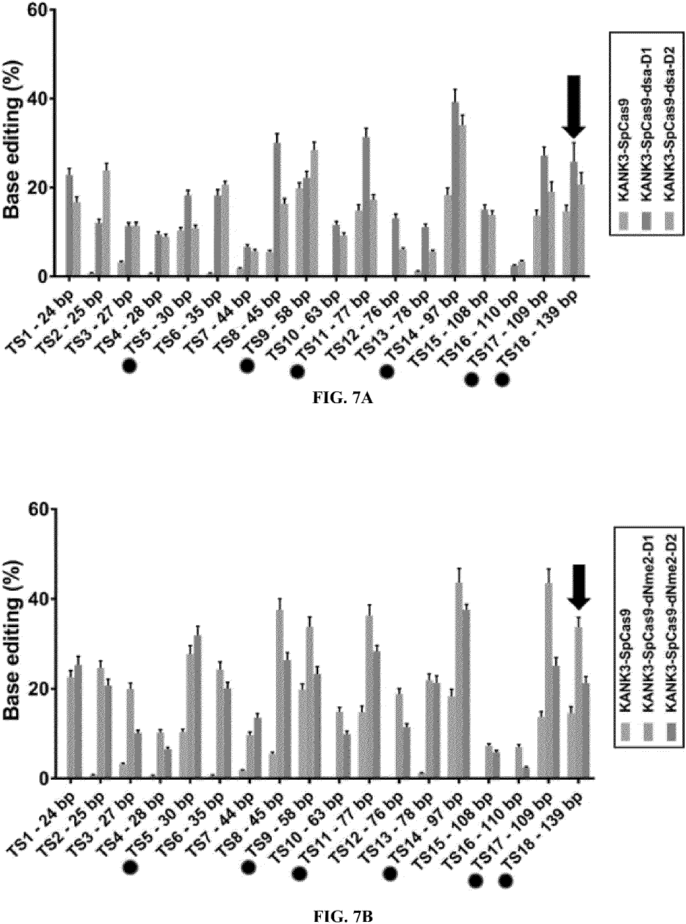

At non-canonical NHG PAM target sites, Cas9 base editor variants display little activity (e.g., SpCas9, SpCas9-NG and xCas9). FIG. 6. Cas9-Cas9 fusion proteins provided favorable activity, in particular, for the D1 orientation of the target sites. The Cas9-ZFP fusions and the Cas9-TALE fusions perform particularly well with regards to high base conversion activity at the NGH PAM target sites. The forty two target sites that were chosen across the three genomic loci also provide information on the proximity of the binding sites of the pDBD or dCas9 to the linked nSpyCas9 base editor with regards to the enhancement in activity. The data for the nSpyCas9-dSauCas9 or the nSpyCas9-dNme2Cas9 across target sites within the KANK3 locus show that there is appreciable enhancement in activity for the Cas9-Cas9 fusions relative to the SpCas9 BE4 for binding sites that have up to 139 bp distance in separation. FIG. 7. Thus, the separation between binding sites where enhancement can be achieved may be similar to the Cas9-Cas9 nuclease platform, which is on the order of 200 bp between the target sequences¹².

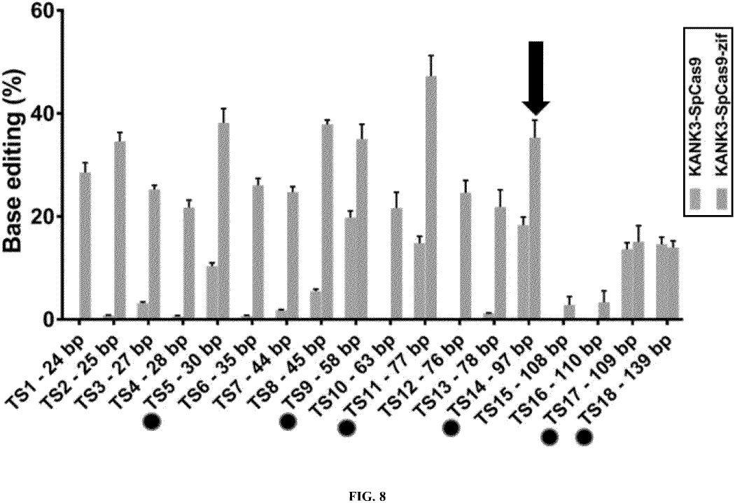

A similar picture emerges for the analysis for the base editing activity of the nSpyCas9-ZFP BE4 construct relative to the SpCas9 BE4 across target sites within the KANK3 locus. The data for the nSpyCas9-ZFP BE4 shows that there is appreciable enhancement in activity for binding sites that have ~ 100 bp distance in separation. FIG. 8. Thus, the enhancement which can be achieved for the nSpyCas9-ZFP system appears to be more modest than the Cas9-Cas9 BE4 system, but the enhancement in base editing activity for the nSpyCas9-ZFP BE4 is more robust than for the Cas9-Cas9 BE4 system. Thus the Cas9-pDBD and Cas9-Cas9 cytosine base editors have a broader targeting range than any of the published Cas9 variant systems and also achieve higher base editing activity. In one embodiment, these frameworks further comprise adenine base editor systems.

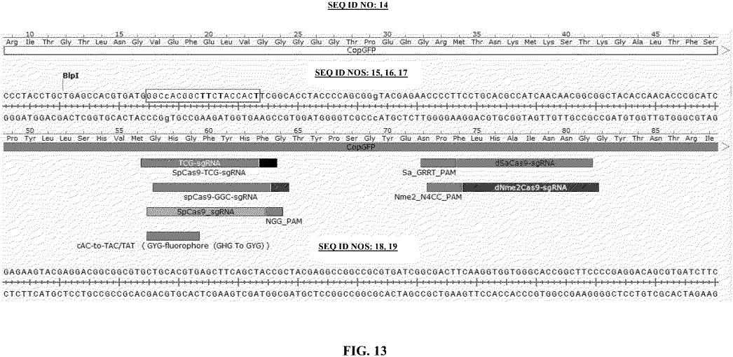

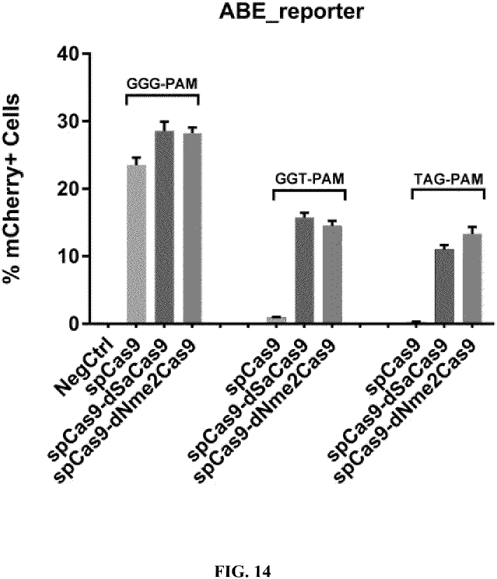

A single copy cytosine base editor reporter (CBE reporter) transgene was generated in HEK293T cells to evaluate the efficiency of cytosine base editors (CBE) that target different PAM sequences. FIG. 13. This transgene contains a single C to T mutation that converts a Tyrosine (TAC) to a Histidine (CAC). The resulting reporter fluoresces blue (CFP). Conversion of the codon back to TAC shifts the emission wavelength to green (GFP). On the coding strand are denoted three different SpCas9 target sequences: one sequence with an optimal PAM [NGG], and two sequences shifted by a single base pair that harbor suboptimal PAMs [NGC or NCG]. Also denoted are neighboring binding sites for other Cas9 orthologs [SauCas9 or Nme2Cas9] that can be utilized as nuclease-dead modules in the context of SpyCas9-dSau/dNme2Cas9 cytosine base editors to localize them to the target site. Base conversion of cytosine to uracil (thymine analog) on the complementary strand will revert the CAC codon to TAC to change the color of the cells from blue to green, which permits a sensitive measure of the base editing rates. Utilizing the adenine base editor reporter (ABE reporter) HEK293T line the efficiency of three different adenine base editor (ABE) constructs were evaluated: 1) SpCas9 ABE, 2) SpCas9-dSaCas9 ABE, and 3) SpCas9-dNme2Cas9 ABE. FIG. 14. These were programmed with three different guide RNAs compatible with three different PAMs for SpCas9 gGG, gGT and tAG -the latter two of which are suboptimal. For the SpCas9-dSaCas9 or SpCas9-dNme2Cas9 ABEs additional guide RNAs were included to target the nuclease-dead orthogonal Cas9 to the indicated binding site in the ABE reporter sequence. FIG. 13. ABEs and their guides were delivered as expression plasmids by transient transfection (800 ng ABE vectors and 200 ng sgRNAs, 150k cells). Adenine conversion rate within the reporter cells was determined by FACS analysis based on the fraction of mCherry positive cells after 3 days. All three ABEs efficiently utilized the NGG PAM to correct the C to T mutation in the ABE reporter. However, only the SpCas9-dSa/dNme2Cas9 ABEs were able to efficiently utilize the NGT or NAG PAMs to achieve reporter correction.

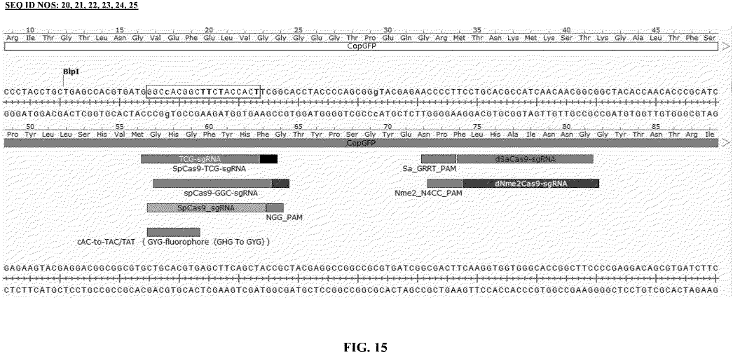

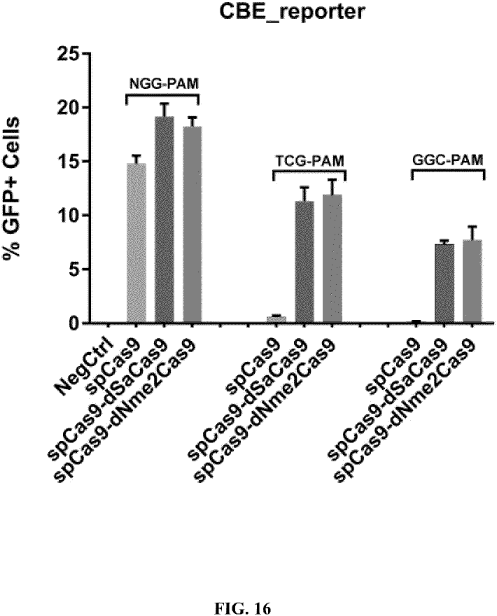

A single copy cytosine base editor reporter (CBE reporter) transgene was generated in HEK293T cells to evaluate the efficiency of cytosine base editors (CBE) that target different PAM sequences. FIG. 15. This transgene contains a single C to T mutation that converts a Tyrosine (TAC) to a Histidine (CAC). The resulting reporter fluoresces blue (CFP). Conversion of the codon back to TAC shifts the emission wavelength to green (GFP). On the coding strand are denoted three different SpCas9 target sequences: one sequence with an optimal PAM [NGG], and two sequences shifted by a single base pair that harbor suboptimal PAMs [NGC or NCG]. Also denoted are neighboring binding sites for other Cas9 orthologs (e.g., SauCas9 or Nme2Cas9) that can be utilized as nuclease-dead modules in the context of SpyCas9-dSau/dNme2Cas9 cytosine base editors to localize them to the target site. Base conversion of cytosine to uracil (thymine analog) on the complementary strand will revert the CAC codon to TAC to change the color of the cells from blue to green, which permits a sensitive measure of the base editing rates. Utilizing the cytosine base editor reporter (CBE reporter) HEK293T line the efficiency of three different cytosine base editor (CBE) constructs were evaluated: 1) SpCas9 CBE, 2) SpCas9-dSaCas9 CBE, and 3) SpCas9-dNme2Cas9 CBE. FIG. 16. These were programmed with three different guide RNAs compatible with three different PAMs for SpCas9 cGG, gGC and tCG - the latter two of which are suboptimal. For the SpCas9-dSaCas9 or SpCas9-dNme2Cas9 CBEs additional guide RNAs were included to target the nuclease-dead orthogonal Cas9 to the indicated binding site in the CBE reporter sequence. FIG. 15. CBEs and their guides were delivered as expression plasmids by transient transfection (800 ng CBE vectors and 200 ng sgRNAs, 150k cells). Cytosine conversion rate within the reporter cells was determined by FACS analysis based on the fraction of GFP positive cells after 3 days. All three CBEs efficiently utilized the NGG PAM to correct the T to C mutation in the CBE reporter. However, only the SpCas9-dSa/dNme2Cas9 CBEs were able to efficiently utilize the NGC or NCG PAMs to achieve reporter correction.

### III. MECP2 Gene Base-Editing Strategies To Treat Rett Syndrome

In one embodiment, the present invention contemplates a sequence-specific base editor (BE)³⁸. Although it is not necessary to understand the mechanism of an invention it is believed that the sequence-specific BE provides a direct reversion of common pathogenic mutations.

It has been reported that pathogenic mutations in the MECP2 gene account for about half of the disease alleles that are associated with this locus²⁷. These lesions are most often reported to be C - T base transitions that produce either a missense or nonsense mutation. Table 1.

Table 1

Representative MECP2 Mutations

| Mutation | Result | AA Change | Mutation Freq (%) | Bystander adenines |
|---|---|---|---|---|
| c.473C>T | Missense | T158M | 8.81 | yes |
| c.502C>T | Nonsense | R168X | 7.63 | no |
| c.763C>T c.763C>T | Nonsense | R255X | 6.68 | no |
| c.808C>T | Nonsense | R270X | 5.80 | no |
| c.916C>T | Missense | R306C | 5.17 | no |
| c.880C>T | Nonsense | R294X | 5.00 | yes |
| c.397C>T | Missense | R133C | 4.56 | yes |
| c.316C>T | Missense | R106W | 2.77 | no |

Five of the eight most common Rett mutations would be suitable targets for adenine base editors in that they that do not have bystander adenines in danger of introducing new missense mutations at neighboring base pairs upon ABE treatment. Of these five suitable targets, the c.808C>T and c.316C>T mutations are targetable with standard SpyCas9 ABEs. The c.502C>T, c.763C>T and c.916C>T mutations are addressable with a Cas9-DBD ABEs.

In principle, an adenine base editor (ABE)¹⁸ should be capable of reverting all eight of the common pathogenic MECP2 mutations, since it can drive T to C transitions in the context of a base pair. Implementation of the current generation of ABEs takes into account: 1) a complementary PAM at the correct position and on the desired DNA strand to allow base conversion, as ABEs have maximal activity on the ssDNA strand within a window roughly 13 to 16 nucleotides 5’ of the PAM element¹⁸, and 2) the absence of nearby adenines on the same strand (e.g., bystanders) that would also fall within the ABE active window, where their conversion to G would promote the generation of a missense mutation.

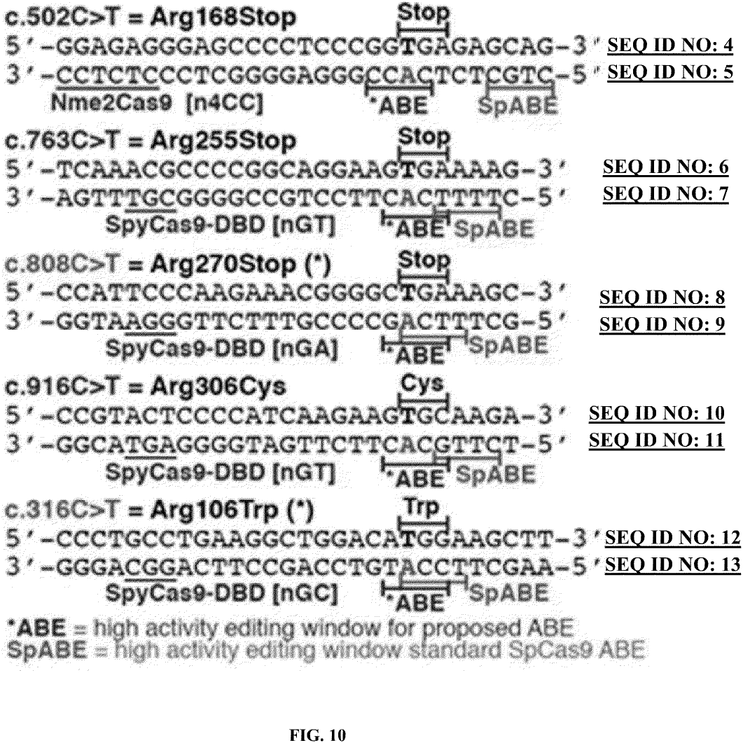

Four of the top five most frequent MECP2 mutations in Rett patients (R168X, R255X, R270X and R306C), which account for ~25% of all pathogenic mutations, do not have bystander concerns. Table 1. However, for SpyCas9 with its NGG PAM, only one out of these four mutant sequences (R270X) is targetable in the “sweet spot” of the ABE. FIG. 10. This fact highlights the importance of the density of available target sites for the Cas9 module within the ABE. The PAM recognition domain of SpyCas9 is a limitation that prevents maximal reversion efficiencies for many common MECP2 mutations. To address the issue of target density, an xCas9 base editor has been engineered to utilize an NGN PAM^{36,} ⁴³, but independent studies using the xCas9 BE framework observed low base conversion at most NGN target sites.⁴³

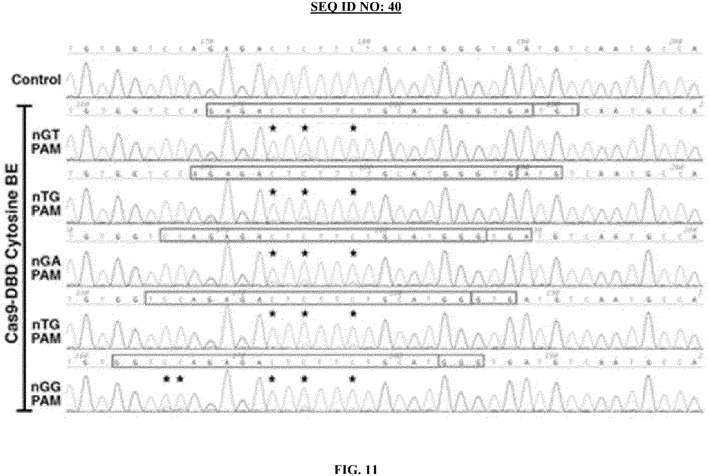

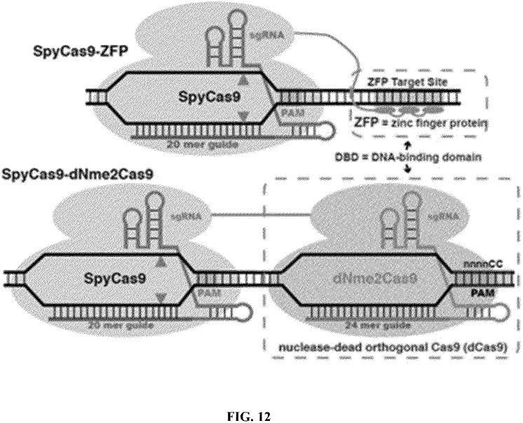

Cas9-DNA-binding domain (Cas9-DBD) base editing platforms have been developed that have a much broader targeting range for PAM recognition than the standard SpyCas9 systems - effectively requiring only a single G within the PAM (NGN or NNG PAM) for function. FIG. 11. This more flexible BE platform is constructed based on an improved SpCas9 nuclease system with broader targeting range and specificity that employs a fusion to a programmable DNA-binding domain (either a Cys2-His2 zinc finger protein²⁵ (ZFP) or an orthogonal nuclease-dead Cas9 (dCas9) to drive genome-locus-specific activity of the nuclease. FIG. 12.

### IV. Development And Characterization Of Cas9-pDBD And Cas9-Cas9 Base Editors

In one embodiment, the present invention contemplates a fusion protein comprising an adenine or cytidine deaminase, a Cas9/sgRNA complex and a programmable DNA binding domain or a Cas9 base editor. In one embodiment, the pDBD base editor is an adenine base editor (ABE). In one embodiment, the pDBD base editor is a cytosine base editor (CBE).

### A. Enhanced Adenine And Cytosine Base Editors (CBEs)

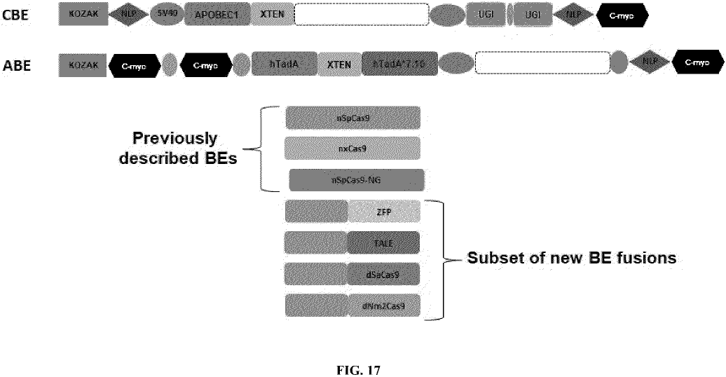

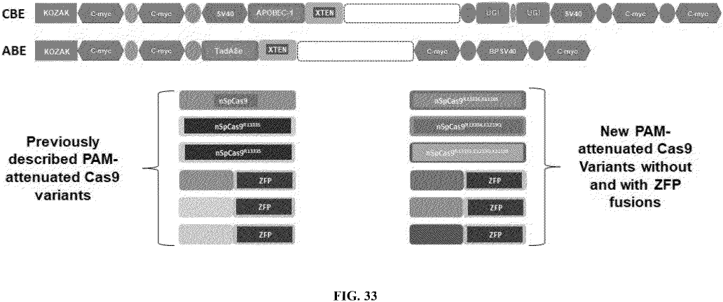

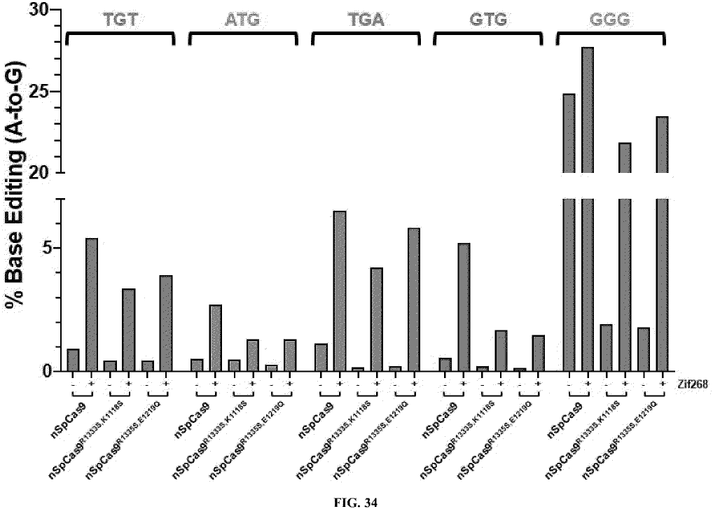

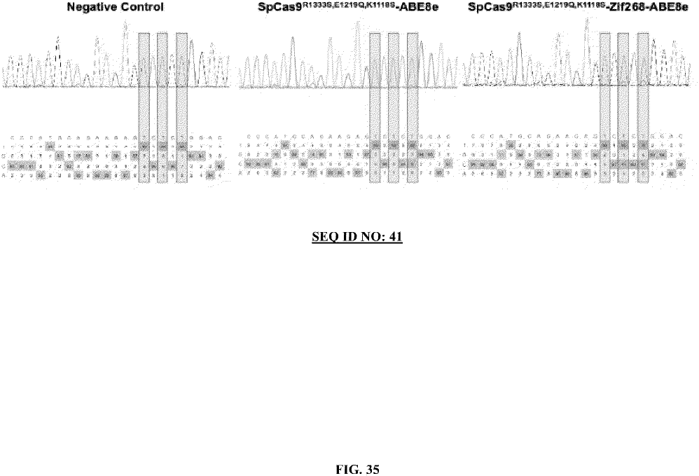

Conventionally, adenine and cytosine base editors are reported to comprise proteins such as, nickase SpCas9, nickase xCas9 or nSpCas9-NG. In one embodiment, the present invention contemplates fusion proteins comprising a Cas9/sgRNA complex and an enhanced adenine and cytosine base editors that include, but are not limited to, zinc finger proteins (ZFP), transcription activator-like effector (TALE) proteins, dead SaCas9 or dead Nm2Cas9. In one embodiment, the fusion protein is flanked by accessory proteins or domains including, but not limited to, adenine deaminase (hTadA-XTEN-hTadA*7.10, TadA8e) or cytidine deaminase (APOBEC1), nuclear localization signal (NLS) sequences (e.g., C-myc or SV40 NLS), intervening linkers (e.g., XTEN or other sequences) and/or uracil glycosylase inhibitor (UGI) proteins. See, FIGS. 17 and 33.

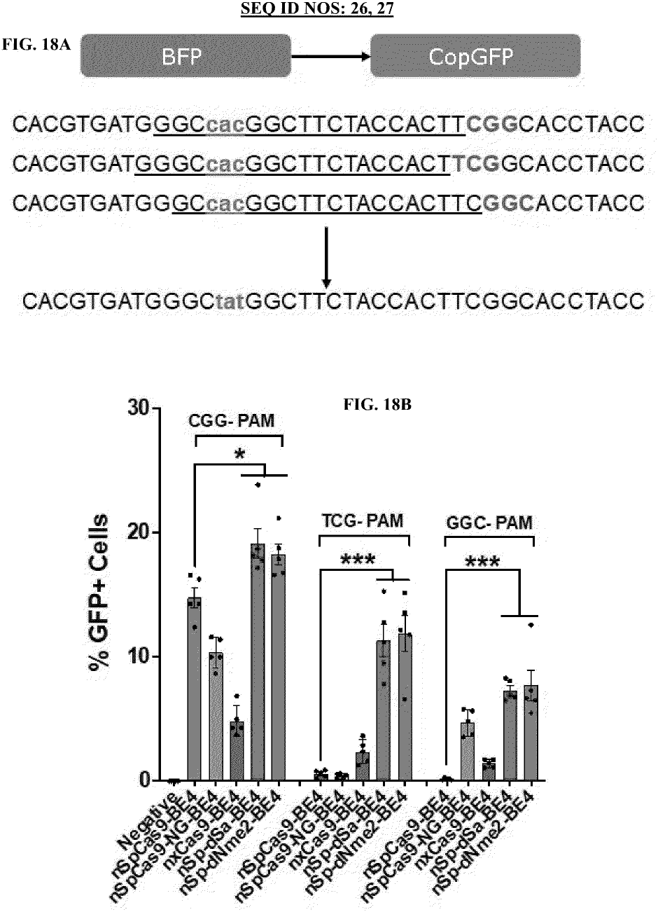

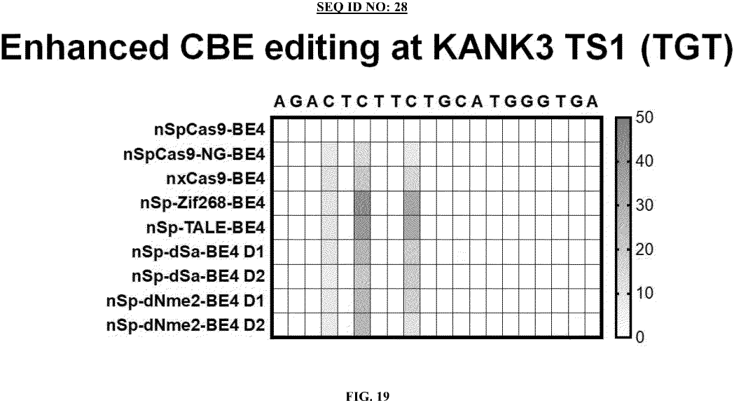

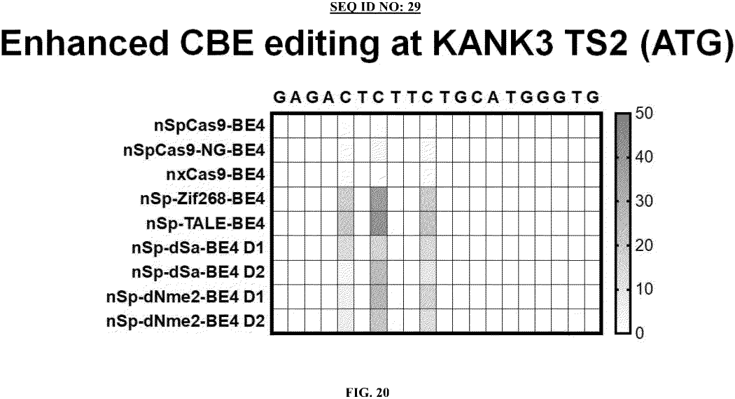

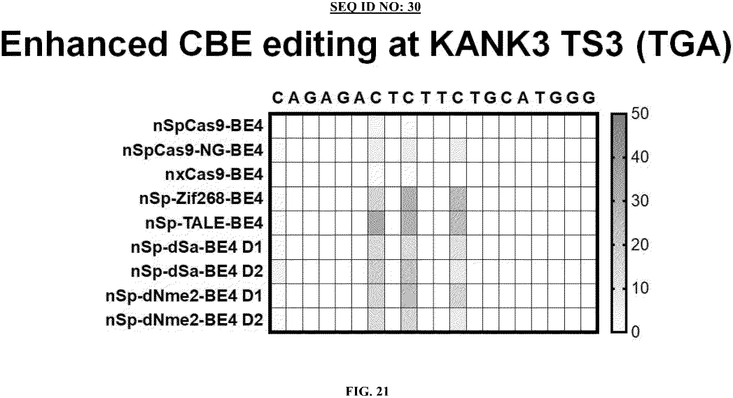

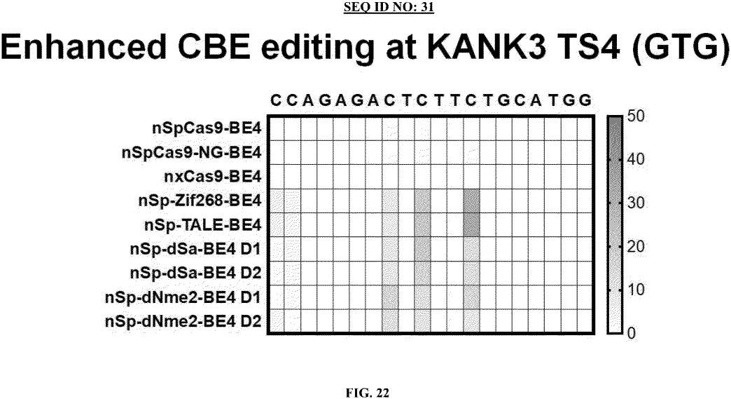

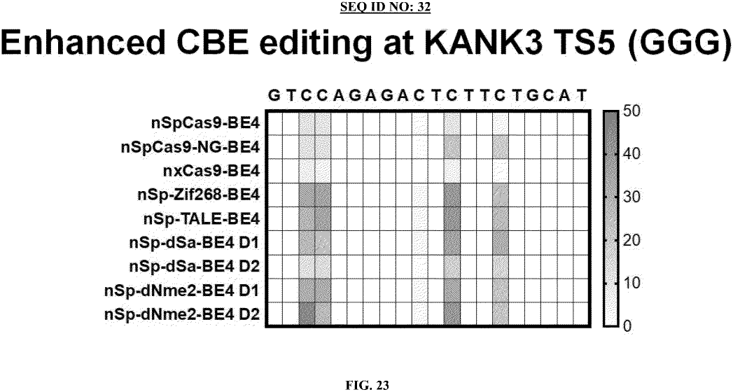

The improved activity of enhanced cytosine base editor embodiments were validated using a CopGFP reporter line. This reporter line shifts from a blue signal (BFP) to a green signal (GFP) subsequent to the modification of the trinucleotide target sequence from “cac” to “tat”. See, FIG. 18A. The data shows that the enhanced CBEs (blue/orange bars) contemplated herein have an approximate 2-fold increase in GFP fluorescence at target sites containing a PAM with a single G in comparison to previously reported CBEs (gray/turquoise/green bars). See, FIG. 18B. Similar data showing improved activity for enhanced CBEs as contemplated herein versus conventional CBE’s has been collected at KANK3 target sites having a variety of PAM sequences: i) TGT (FIG. 19); ii) ATG (FIG. 20); iii) TGA (FIG. 21); iv) GTG (FIG. 22); v) GGG (FIG. 23).

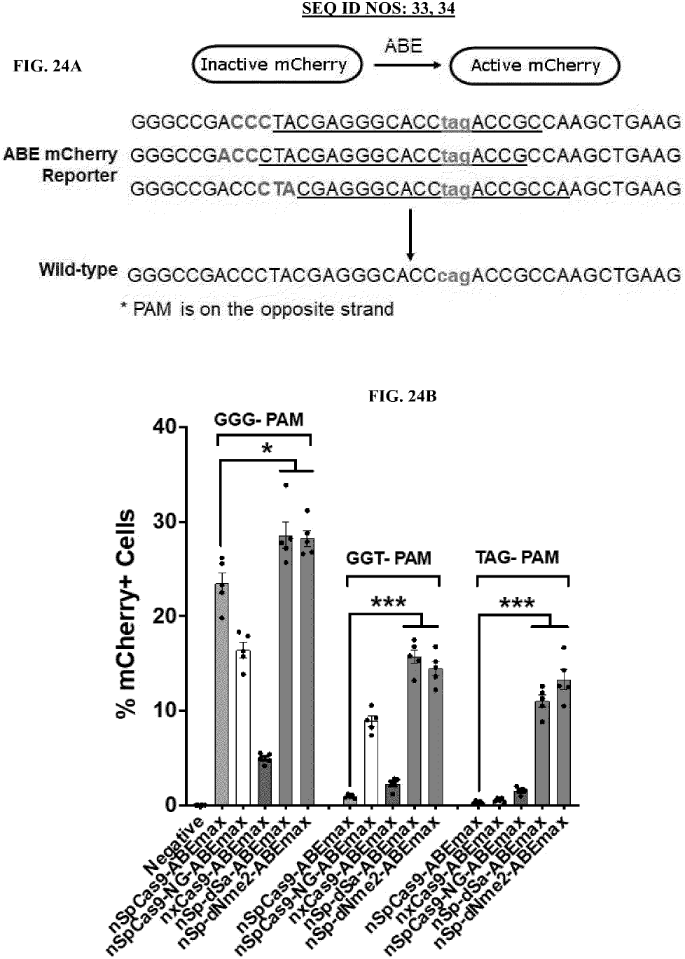

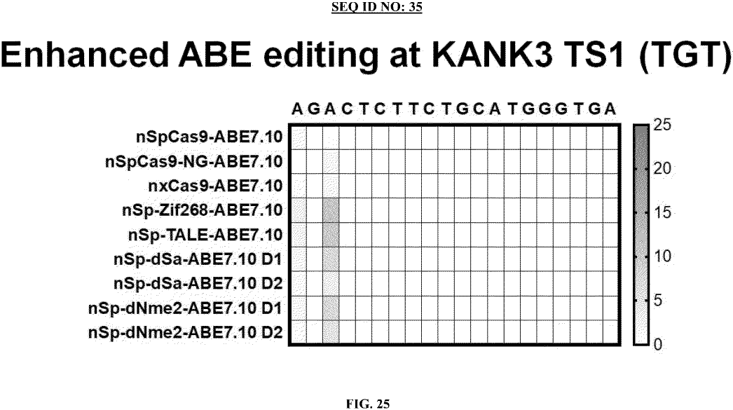

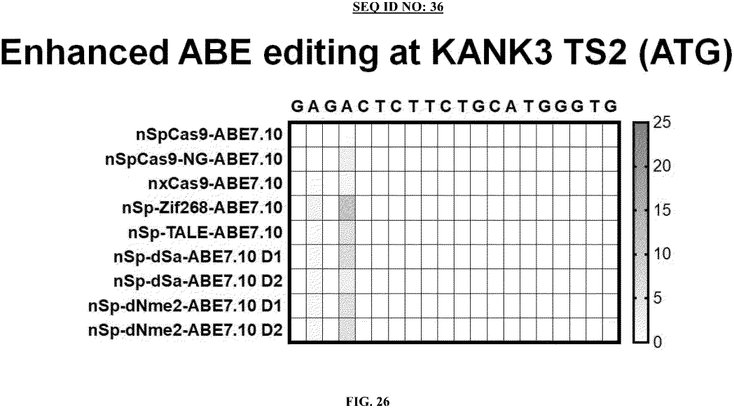

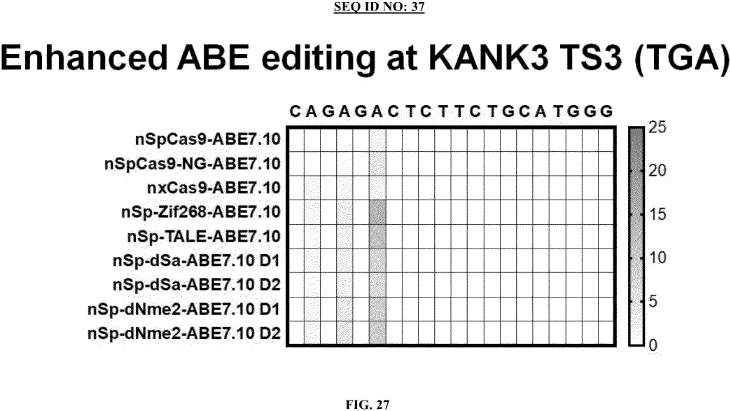

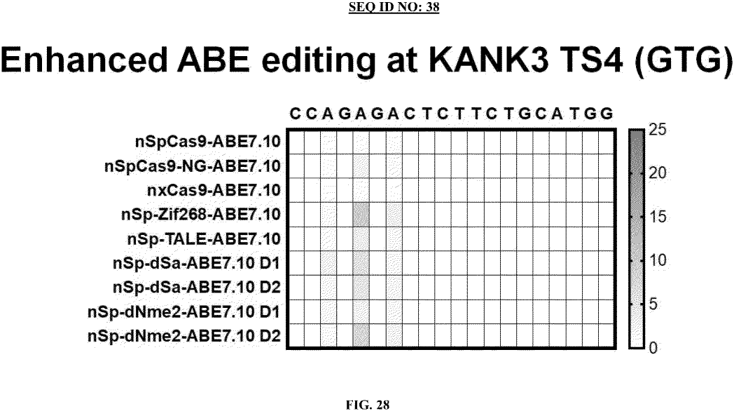

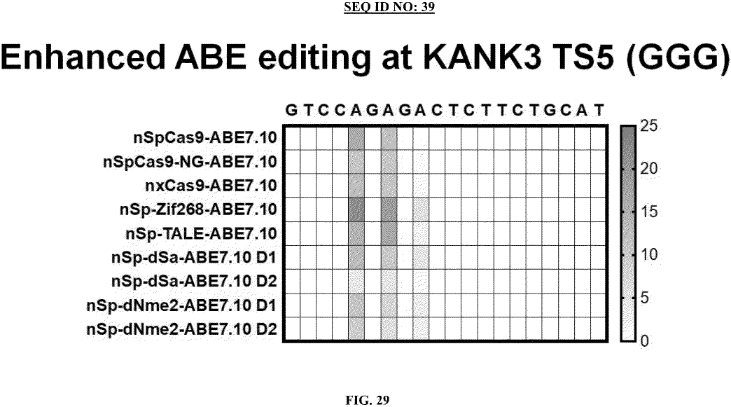

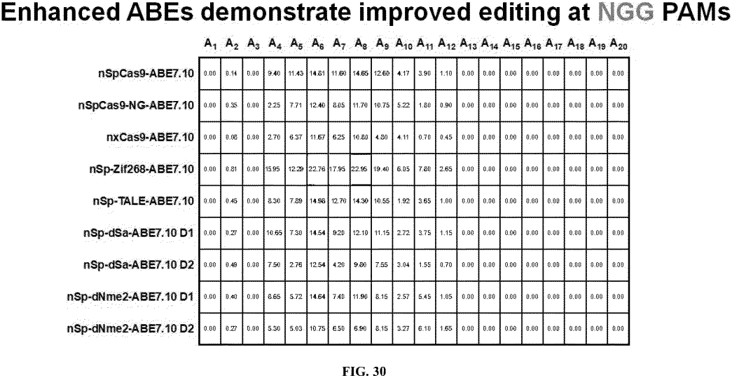

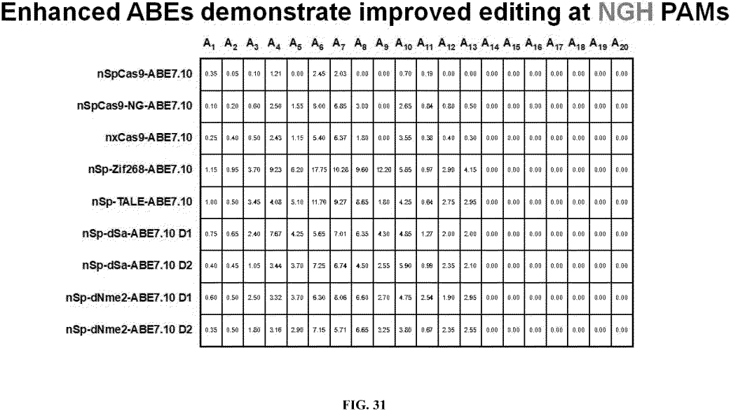

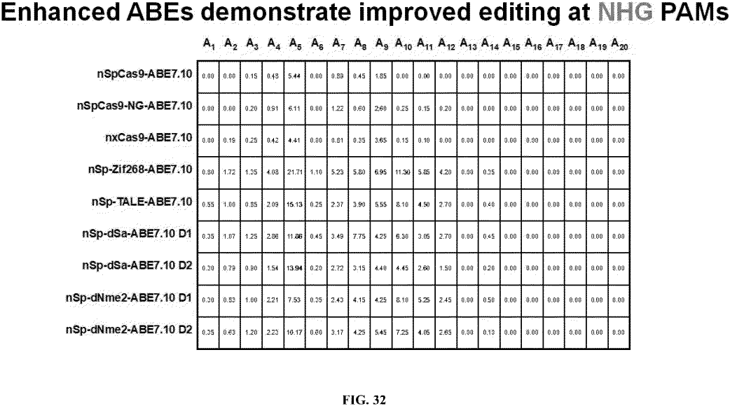

The improved activity of enhanced adenine base editor embodiments were validated using an mCherry reporter line. This reporter line shifts from no signal to a red signal subsequent to the modification of the codon target sequence from “tag” to “cag”. See, FIG. 24A. The data shows that the enhanced ABEs (blue/orange bars) contemplated herein have an approximate 2-fold increase in GFP fluorescence at target sites containing a PAM with a single G in comparison to previously reported ABEs (gray/white/green bars). See, FIG. 24B. Similar data showing improved activity for enhanced ABEs as contemplated herein versus conventional ABE’s has been collected at KANK3 target sites having a variety of PAM sequences: i) TGT (FIG. 25): ii) ATG (FIG. 26); iii) TGA (FIG. 27); iv) GTG (FIG. 28); v) GGG (FIG. 29); vi) NGG (FIG. 30); viii) NGH (FIG. 31); ix) NHG (FIG. 32).

### V. Attenuated Cas9 Proteins

Although it is not necessary to understand the mechanism of an invention, it is believed that an attenuated nSpyCas9 system provides an avenue to dramatically reduce the off-target editing rates for any base editing system. In one embodiment, these base editing constructs target pathogenic mutations. In particular, the PAM recognition domain has a reduced affinity for the cognate PAM of a specific Cas9 protein. It is believed that this attenuation facilitates pDBD-mediated discrimination of binding between target and non-cognate target sites as described herein. Previous reporting has identified that, in the SpyCas9 an R1333S or R1335S substitution may result in attenuated Cas9 binding to the cognate PAM.

In one embodiment, an attenuated Cas9 protein comprises an amino acid substitution. In one embodiment, the amino acid substitution is in the PAM recognition domain. In one embodiment, the amino acid substitution comprises R1333S and K1118S. In one embodiment, the amino acid substitution comprises R1335K and E1219Q. In one embodiment, the amino acid substitution comprises R1333S, E1219Q and K1118S. In one embodiment, the attenuated Cas9 protein further comprises a pDBD protein. In one embodiment, the pDBD protein is a zinc finger protein. See, FIG. 33.

### REFERENCES

1. Komor, A. C., Kim, Y. B., Packer, M. S., Zuris, J. A. & Liu, D. R. Programmable editing of a target base in genomic DNA without double-stranded DNA cleavage. Nature (2016).
2. Nishida, K. et al. Targeted nucleotide editing using hybrid prokaryotic and vertebrate adaptive immune systems. Science (2016). doi:10.1126/science.aaf8729
3. Gaudelli, N. M. et al. Programmable base editing of A·T to G·C in genomic DNA without DNA cleavage. Nature 551, 464-471 (2017).
4. Rees, H. A. & Liu, D. R. Base editing: precision chemistry on the genome and transcriptome of living cells. Nat Rev Genet 70, 3240 (2018).
5. Kuscu, C. et al. CRISPR-STOP: gene silencing through base-editing-induced nonsense mutations. Nature Methods (2017). doi:10.1038/nmeth.4327
6. Gapinske, M. et al. CRISPR-SKIP: programmable gene splicing with single base editors. Genome Biol 19, 107 (2018).
7. Hu, J. H. et al. Evolved Cas9 variants with broad PAM compatibility and high DNA specificity. Nature (2018). doi:10.1038/nature26155
8. Nishimasu, H. et al. Engineered CRISPR-Cas9 nuclease with expanded targeting space. Science (2018). doi:10.1126/science.aas9129
9. Bolukbasi, M. F. et al. DNA-binding-domain fusions enhance the targeting range and precision of Cas9. Nature Methods 12, 1150-1156 (2015).
10. Persikov, A. V. et al. A systematic survey of the Cys2His2 zinc finger DNA-binding landscape. Nucleic Acids Research 43, 1965-1984 (2015).
11. Miller, J. C. et al. Improved specificity of TALE-based genome editing using an expanded RVD repertoire. Nature Methods 12, 465-471 (2015).
12. Bolukbasi, M. F. et al. Orthogonal Cas9-Cas9 chimeras provide a versatile platform for genome editing. Nature Communications 9, 4856 (2018).
13. Edraki, A. et al. A Compact, High-Accuracy Cas9 with a Dinucleotide PAM for In Vivo Genome Editing. Molecular Cell (2018). doi:10.1016/j.mo1ce1.2018.12.003
14. Yeh, W.-H., Chiang, H., Rees, H. A., Edge, A. S. B. & Liu, D. R. In vivo base editing of postmitotic sensory cells. Nature Communications 9, 2184 (2018).
15. Liang, P. et al. Genome-wide profiling of adenine base editor specificity by EndoV-seq. Nature Communications 10, 67 (2019).
16. Kim, D. et al. Genome-wide target specificities of CRISPR RNA-guided programmable deaminases. Nature biotechnology (2017). doi:10.1038/nbt.3852
17. Gehrke, J. M. et al. An APOBEC3A-Cas9 base editor with minimized bystander and off-target activities. Nature Biotechnology (2018). doi:10.1038/nbt.4199
18. Komor, A. C. et al. Improved base excision repair inhibition and bacteriophage Mu Gam protein yields C:G-to-T:A base editors with higher efficiency and product purity. Sci Adv 3, eaao4774 (2017).
19. Pavletich, N. P. & Pabo, C. O. Zinc finger-DNA recognition: crystal structure of a Zif268-DNA complex at 2.1 A. Science 252, 809-817 (1991).
20. Wolfe, S. A., Greisman, H. A., Ramm, E. I. & Pabo, C. O. Analysis of zinc fingers optimized via phage display: evaluating the utility of a recognition code. J Mol Biol 285, 1917-1934 (1999).
21. Cermak, T. et al. Efficient design and assembly of custom TALEN and other TAL effector-based constructs for DNA targeting. Nucleic Acids Research 39, e82-e82 (2011).
22. Ran, F. A. et al. In vivo genome editing using Staphylococcus aureus Cas9. Nature 520, 186-191 (2015).

We claim:

1. A method, comprising;
a) providing;
i) a nucleic acid sequence encoding at least one mutated base pair; and
ii) a fusion protein comprising a Cas9/sgRNA complex, a programmable DNA binding domain (pDBD) and an adenine base editor (ABE) protein or a cytosine base editor (CBE) protein;
b) contacting said fusion protein with said mutated base pair; and
c) reverting said base pair to a wild type base pair.

2. The method of claim 1, wherein said programmable DNA binding domain is a zinc finger protein (ZFP), a transcription activator-like effector (TALE) protein or an orthogonal dCas9/sgRNA complex.

3. The method of claim 1, wherein said fusion protein further comprises a cytidine deaminase protein or an adenine deaminase protein.

4. The method of claim 1, wherein said at least one mutated base pair is an MECP2 gene mutation.

5. The method of claim 1, further providing a biological sample comprising said at least one mutated base pair.

6. The method of claim 5, wherein said biological sample is a human biological sample.

7. The method of claim 1, further comprising administering said fusion protein to a patient exhibiting at least one symptom of a genetic disease.

8. The method of claim 7, further comprising reducing said at least one symptom of said genetic disease with said fusion protein.

9. The method of Clam 7, wherein said genetic disease is Rett syndrome.

10. The method of claim 1, wherein said adenine base editor or said cytosine base editor hybridizes proximate to a protospacer adjacent motif (PAM) containing a single G.

11. The method of claim 1, wherein said adenine base editor or said cytosine base editor hybridizes to a protospacer adjacent motif that is non-canonical for said Cas9/sgRNA complex.

12. The method of claim 1, wherein said fusion protein is selected from the group consisting of a CBE/ABE-nSpyCas9-ZFP fusion protein, a CBE/ABE-nSpyCas9-TALE fusion protein and a CBE/ABE-nSpyCas9-dSauCas9/dNme2Cas9 fusion protein.

14. The method of claim 1, wherein said reverting comprises a base conversion activity that has a two-fold greater efficiency than a standard base editor protein lacking a pDBD.

15. A composition comprising a Cas9/sgRNA framework attached to a programmable DNA binding domain and an adenine or a cytosine base editor protein that hybridizes proximate to a single G protospacer adjacent motif containing a single G.

16. The composition of claim 15, wherein said Cas9/sgRNA framework further comprises an adenine deaminase protein or a cytidine deaminase protein.

17. The composition of claim 15, wherein said programmable DNA binding domain is a zinc finger protein or an orthogonal dCas9/sgRNA complex.

18. The composition of claim 15, wherein said Cas9 has attenuated DNA binding affinity to a protospacer adjacent motif containing a dual G.

19. An attenuated Cas9 protein comprising a PAM recognition domain having at least two amino acid substitutions, wherein said PAM recognition domain has an attenuated affinity for its cognate PAM sequence.

20. The attenuated Cas9 protein of claim 19, wherein said at least two amino acid substitutions are R1333S and K1118S.

21. The attenuated Cas9 protein of claim 19, wherein said at least two amino acid substitutions are R1335K and E1219Q.

22. The attenuated Cas9 protein of claim 19, wherein said at least two amino acid substitutions are R1333S, E1219Q and K1118S.

23. The attenuated Cas9 protein of claim 19, wherein said attenuated Cas9 protein is attached to a pDBD protein.

24. The attenuated Cas9 protein of claim 19, wherein said pDBD protein is a zinc finger protein, a transcription activator-like effector (TALE) protein or a Cas9 protein.
